SEQUENCE LISTING (25 sequences)

SEQ ID NO: 1 - protein, 5 aa, synthetic construct
  REGION 1..5: note = Humanized antibody sequence
SYLIH

SEQ ID NO: 2 - protein, 17 aa, synthetic construct
  REGION 1..17: note = Humanized antibody sequence
VINPRSGDSHYNEKFKD

SEQ ID NO: 3 - protein, 6 aa, synthetic construct
  REGION 1..6: note = Humanized antibody sequence
SDYGAY

SEQ ID NO: 4 - protein, 11 aa, synthetic construct
  REGION 1..11: note = Humanized antibody sequence
RASQSISNNLH

SEQ ID NO: 5 - protein, 7 aa, synthetic construct
  REGION 1..7: note = Humanized antibody sequence
YASQSAF

SEQ ID NO: 6 - protein, 9 aa, synthetic construct
  REGION 1..9: note = Humanized antibody sequence
QQSNSWPHT

SEQ ID NO: 7 - protein, 115 aa, synthetic construct
  REGION 1..115: note = Humanized antibody sequence
QVQMQQSGAEVKKPGASVKVSCKASGYAFTSYLIHWIKQRPGQGLEWIGVINPRSGDSHYNEKFKDRVTMTADQSISTAYMELSRLRSDDTAVYYCARSDYGAYWGQGTLVTVSS

SEQ ID NO: 8 - protein, 107 aa, synthetic construct
  REGION 1..107: note = Humanized antibody sequence
EIVLTQSPATLSVSPGERATLSCRASQSISNNLHWYQQKPGQAPRLLIKYASQSAFGIPARFSGSGSGTEFTLTISSLQSEDFAVYFCQQSNSWPHTFGQGTKLEIK

SEQ ID NO: 9 - DNA, 15 bp, synthetic construct
  misc_feature 1..15: note = Humanized antibody sequence
  source 1..15: mol_type = other DNA
agttacttgatacac

SEQ ID NO: 10 - DNA, 51 bp, synthetic construct
  misc_feature 1..51: note = Humanized antibody sequence
  source 1..51: mol_type = other DNA
gtgattaatcctagaagtggtgatagtcactacaatgagaagttcaaggac

SEQ ID NO: 11 - DNA, 18 bp, synthetic construct
  misc_feature 1..18: note = Humanized antibody sequence
  source 1..18: mol_type = other DNA
tcagattacggggcttac

SEQ ID NO: 12 - DNA, 33 bp, synthetic construct
  misc_feature 1..33: note = Humanized antibody sequence
  source 1..33: mol_type = other DNA
agggccagccaaagtattagcaacaacctacac

SEQ ID NO: 13 - DNA, 21 bp, synthetic construct
  misc_feature 1..21: note = Humanized antibody sequence
  source 1..21: mol_type = other DNA
tatgcttcccagtccgctttt

SEQ ID NO: 14 - DNA, 27 bp, synthetic construct
  misc_feature 1..27: note = Humanized antibody sequence
  source 1..27: mol_type = other DNA
caacagagtaacagctggcctcacacg

SEQ ID NO: 15 - DNA, 345 bp, synthetic construct
  misc_feature 1..345: note = Humanized antibody sequence
  source 1..345: mol_type = other DNA
caggtccagatgcagcagtctggagctgaggtgaagaagcctggggcctcagtgaaggtttcctgcaaggcttctggatacgccttcactagttacttgatacactggattaaacagaggcctggacagggccttgagtggattggagtgattaatcctagaagtggtgatagtcactacaatgagaagttcaaggacagggtcacaatgactgcagaccagtccatcagcactgcctacatggagctcagcaggctgagatctgatgacacggcggtctattactgtgcaagatcagattacggggcttactggggccaagggactctggtcaccgtctcctca

SEQ ID NO: 16 - DNA, 321 bp, synthetic construct
  misc_feature 1..321: note = Humanized antibody sequence
  source 1..321: mol_type = other DNA
gaaattgtgttgactcagtcaccagccaccctgtctgtgtctccaggagaaagagccaccctttcctgcagggccagccaaagtattagcaacaacctacactggtatcagcagaaacctggccaggctccaaggcttctcatcaaatatgcttcccagtccgcttttggcatccccgccaggttcagtggcagtggatcagggacagaattcactctcaccatcagcagtctgcagtctgaagattttgcagtttatttctgtcaacagagtaacagctggcctcacacgttcggtcaggggaccaagctggagatcaaa

SEQ ID NO: 17 - protein, 513 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 18 - protein, 334 aa, Homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 19 - protein, 584 aa, Homo sapiens
MKMASSLAFLLLNFHVSLFLVQLLTPCSAQFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPWIAALAGTLPISLLLLAGASYFLWRQQKEKIALSRETEREREMKEMGYAATEQEISLREKLQEELKWRKIQYMARGEKSLAYHEWKMALFKPADVILDPDTANAILLVSEDQRSVQRAEEPRDLPDNPERFEWRYCVLGCENFTSGRHYWEVEVGDRKEWHIGVCSKNVERKKGWVKMTPENGYWTMGLTDGNKYRALTEPRTNLKLPEPPRKVGIFLDYETGEISFYNATDGSHIYTFPHASFSEPLYPVFRILTLEPTALTICPIPKEVESSPDPDLVPDHSLETPLTPGLANESGEPQAEVTSLLLPAHPGAEVSPSATTNQNHKLQARTEALY

SEQ ID NO: 20 - protein, 14 aa, Homo sapiens
HLFPTMSAETMELK

SEQ ID NO: 21 - protein, 444 aa, synthetic construct
  REGION 1..444: note = Humanized antibody
QVQMQQSGAEVKKPGASVKVSCKASGYAFTSYLIHWIKQRPGQGLEWIGVINPRSGDSHYNEKFKDRVTMTADQSISTAYMELSRLRSDDTAVYYCARSDYGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 214 aa, synthetic construct
  REGION 1..214: note = Humanized antibody
EIVLTQSPATLSVSPGERATLSCRASQSISNNLHWYQQKPGQAPRLLIKYASQSAFGIPARFSGSGSGTEFTLTISSLQSEDFAVYFCQQSNSWPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 7 aa, Mus musculus
YASQSIF

SEQ ID NO: 24 - protein, 441 aa, Macaca fascicularis
QFAVVGPPGPILAMVGEDADLPCHLFPTMSAETMELRWVSSNLRQVVNVYADGKEVEDRQSAAYRGRTSILRDGITAGKAALRIHNVTASDSGKYLCYFQDGDFYEKALVELKVAALGSDLHIDVKGYEDGGIHLECRSTGWYPQPQIRWSNDKGENIPAVEAPVFVDGVGLYAVAASVILRGSSGEGVSCTIRSSLLGLEKTTSISIAGPFFRRAQSWIAALAGTLPVLLLLLGGAGYFLWRQQEEKKTLFRKKKREQELRELAWSTVKQEKSTREKLLEEIRWRSMQYTVLGERPSAYNEWKKALFKPADVILDPKTANPILLVSEDQRSVQRAKEPQDLPDNPERFDWHYCVLGCESFMSGRHYWEVEVGDRKEWHIGVCSKNVQRKGWVKMTPENGSHIHTFLDVSFSEPLYPVFRILTLEPTALTICPAPKEEESS

SEQ ID NO: 25 - protein, 21 aa, Homo sapiens
LPCHLFPTMSAETMELKWVSS

(19) United States
(12) Patent Application Publication
(10) Pub. No.: US 2025/0353909 A1
(43) Pub. Date: Nov. 20, 2025

(54) ANTI-BTN3A ANTIBODIES FOR USE IN METHODS OF TREATING GASTRO-INTESTINAL INFLAMMATORY DISORDERS

(71) Applicant: IMCHECK THERAPEUTICS, MARSEILLE (FR)

(72) Inventors: Sophie AGAUGUE, Marseille Cedex 09 (FR); Paul FROHNA, Marseille Cedex 09 (FR)

(21) Appl. No.: 18/862,768

(22) PCT Filed: May 9, 2023
(86) PCT No.: PCT/EP2023/062207

§ 371 Date: Nov. 4, 2024

(30) Foreign Application Priority Data
May 10, 2022 (EP) 22305694.6

(51) Int. Cl. C07K 16/28; A61P 1/04; C07K 16/24

(52) U.S. Cl. CPC C07K 16/2827; A61P 1/04; C07K 16/241; C07K 16/244; C07K 2317/21; C07K 2317/24; C07K 2317/92

(57) ABSTRACT

It is disclosed BTN3A inhibitory antibodies for use in treating gastro-intestinal inflammatory disorders, such as inflammatory bowel disease. The disclosure more specifically relates to specific anti-BTN3A antibodies that specifically bind to BTN3A and inhibit the degranulation of Vγ9/Vδ2 T cells and their use in the manufacturing of novel drugs for use in treating gastro-intestinal inflammatory disorders such as ulcerative colitis and Crohn's disease.

### INTRODUCTION

Gastrointestinal disorders refer to diseases involving the gastrointestinal tract, namely the esophagus, stomach, small intestine, large intestine and rectum, and the accessory organs of digestion, the liver, gallbladder, and pancreas. The term encompasses acute, chronic, recurrent or functional disorders and covers a wide range of diseases of different origins, i.e. congenital, environmental, metabolic, functional, central nervous system processing, bacterial, autoimmune . . . Several large scale worldwide studies have been conducted and it was shown that 24 to 40% of persons worldwide have gastrointestinal disorders which affect quality of life and healthcare use and costs (Sperber A D et al, Gastroenterology, 2021, January; 160(1):99-114.e3; Mathews C et al, Clin Gastroenterol Hepatol, 2021, July 1:S1542-3565(21)00711-4).

Among those gastro-intestinal inflammatory disorders, Inflammatory Bowel Diseases (IBDs) are one of the most impactful and major public health problems. IBDs are debilitating chronic inflammatory disorders of the gastrointestinal tract with the peak age of onset in adolescence and young adulthood. They comprise two idiopathic GI disorders known as ulcerative colitis (UC) and Crohn's disease (CD). Despite intense research efforts, the disease aetiology(ies) is (are) not fully understood. However, it appears that both genetic and environmental factors are involved in IBD causation, affecting the interaction between the intestinal mucosa and luminal bacteria, with a breakdown in the regulatory constraints of mucosal immune responses to enteric bacteria, in other words, an immune (inflammatory) response that is too easily triggered and/or needlessly prolonged. Both UC and CD are chronic disorders of a remitting and relapsing kind. Just as the cause of the initial onset of IBD is unknown, what leads to remission and relapse is also uncertain (Tavakoli P et al, Public Health Rev, 2021, Public Health Rev. 2021 May 5; 42:1603990).

The incidence of IBD is increasing globally and in 2019, the highest reported prevalence values for IBD were in Europe, North America and Australia (Tavakoli P et al, Public Health Rev, 2021, see supra). The incidence of CD has increased by 70% and incidence of UC has increased by 60%, 2017 estimation was at 6 to 8 million people affected worldwide.

UC is characterized by chronic inflammation of the large intestine with abnormal activation of the immune system. It affects the inner-most layer of the colon and rectum. CD can affect any level of the intestinal tract from the mouth to the anus and across all layers of the bowel wall, but mostly affects lower small intestine (ileum) and colon. The most common symptoms of IBD include diarrhea, rectal bleeding, intermittent nausea and vomiting, and abdominal pain or tenderness (Baumgart D C, and Sandborn, WJ, The Lancet, 2007, May 12; 369(9573):1641-57; Strober, W, Fuss, I, and Mannon, P, J Clin Invest, 2007, March; 117(3):514-21). The symptoms are due to intestinal damage resulting from the exaggerated inflammatory response. Complications from these immune-mediated diseases include anemia, malnutrition, bowel obstruction, fistula, infection, and an increased risk of colon cancer. Extra-intestinal manifestations may also develop, such as joint problems (arthralgia, arthritis, and ankylosing spondylitis), rashes and skin conditions (erythema nodosum, psoriasis), chronic liver disease (primary sclerosing cholangitis) and eye conditions (such as uveitis). Clinical management focuses on keeping patients in remission and asymptomatic with a primary aim of reducing inflammation during relapse and secondary aims of prolonging the time spent in endoscopic remission and mucosal healing, but there is currently no cure for IBD patients. The current management options are based on different therapeutic categories: (i) anti-inflammatory drugs like aminosalicylates or steroids, (ii) immune suppressors like aziathropine, cyclosporine, JAK inhibitors or (iii) biologics (anti-TNFa, anti-IL-12/23, anti-a4b7 monoclonal antibodies). Despite improvements in healthcare, IBD still impacts patient's lifespan and recent estimates indicate that IBD reduces life expectancy by 6.6 to 8.1 years in females and 5.0 to 6.1 years in men (Kuenzig Me et al, CMAJ, 2020 Nov. 9; 192(45):E1394-E1402). Thus, there is still a significant unmet medical need (Danese S et al, Dig Dis, 2019, 37(4): 266-83).

In humans and non-human primates (NHPs), the major peripheral γδ T cell subset expresses a T Cell Receptor (TCR) composed of Vγ9 and Vδ2 chains. This Vγ9Vδ2 T cell subset represents about 5% of CD3+ cells in peripheral blood, and more than 80% of the peripheral γδ T cells in healthy adults (Bonneville M et al., Nat Rev Immunol, 2010; Poggi A and Zocchi M R, Front Immunol, 2014). In vitro studies have demonstrated that Vγ9Vδ2 T cells have the capacity to detect stress signals from infected and malignant cells that are associated with intracellular accumulation of organic pyrophosphate-containing molecules called phosphoantigens (pAgs). The identification of pAgs as potent and specific activators of Vγ9Vδ2 T cells led to the understanding that BTN3A plays a mandatory role in the antigenic activation of Vγ9Vδ2 T cells, which was an important advancement in the understanding of Vγ9Vδ2 T cell biology. BTN3A is a member of the butyrophilin family of type 1 transmembrane proteins, which in turn belong to the Ig superfamily. Three isoforms of BTN3A have been described (BTN3A1, BTN3A2 and BTN3A3), which are distinguished by their intracellular domains (e.g., presence or absence of a B30.2/SPRY domain), as well as a small number of amino acid differences within their ectodomains. The intracellular accumulation of pAgs induces a conformational change in BTN3A1 through the interaction of pAgs with the intracellular B30.2 domain. This interaction leads to the specific recognition of BTN3A1 by Vγ9Vδ2 T cells which are subsequently activated (Gu S et al., Semin Cell Dev Biol, 2018, December; 84:65-74 December; 84:65-74). BTN3A1 isoform is the only molecule able to signal intracellular pAg accumulation to the cell surface and to trigger Vγ9Vδ2 T cell recognition and activation (Gu S et al., Semin Cell Dev Biol, 2018, see supra).

BTN3A expression is restricted to humans and non-human primates (NHP). Furthermore, BTN3A orthologs are not expressed in rodents and rodents also lack Vγ9Vδ2 T cells, which make them unsuitable for testing BTN3A/Vγ9Vδ2-based therapies.

Previous work by the McCarthy lab has identified that human intestine contains two distinct subsets of Vδ2 T-cells identified by differential expression of the ‘tissue residency’ marker CD103. In healthy colon, the mucosa is populated largely by CD103+ Vδ2 T-cells that display only weak cytokine responses to microbial PAg, whereas colon from patients with Crohn's disease (CD) is dominated instead by CD103− Vδ2 T-cells that display enhanced inflammatory cytokine production upon PAg exposure in vitro. Furthermore, analyses of blood Vδ2 T-cells from CD patients revealed that their frequency and gut-homing potential in the circulation were associated with differential expression of TRM markers including CD69 and CD27, suggesting that cell recruitment/retention in the gut likely plays a major role in shaping mucosal Vδ2 T-cell activity. In addition, other groups showed that circulating γδ T cells were more abundant in active IBD (CD and UC) and in gut biopsies, Vd2 T cells in particular were more present in biopsies of late than early IBD patients, these Vδ2 T cells producing more IFNδ, TNFα and IL-17 (Giacomelli R et al, Clin Exp Immunol, 1994; Mc Carthy N E et al, J Clin Invest, 2015; Mc Carthy Ne et al, J Immunol, 2013; Markovits N et al, Inflammopharmacology, 2017; Lo Presti E et al, J Crohns Colitis, 2019). Thus, targeting the Vδ2 population by blocking its functions through an anti-BTN3A monoclonal antibody could be of potential therapeutic interest. No γδ-targeted therapy is currently under development for IBD and the unmet medical need is still high.

WO2012080769 describes a specific murine monoclonal antibody referred as mAb 103.2 having the capacity to inhibit the cytolytic function, production and proliferation of Vγ9Vδ2 T cells. Consequently, such murine antibody mAb 103.2, and their corresponding chimeric and humanized versions or fragments thereof were suggested to be potentially useful in treating inflammatory disorders.

WO2020136218 further reports that the Fab fragment of mAb 103.2 exhibit activating properties having the capacity to activate the cytolytic function, production and proliferation of Vγ9Vδ2 T cells.

The present invention now relies on the discovery that certain humanized anti-BTN3A1 antibody can powerfully inhibit Vγ9Vδ2 T cell functions in vitro in healthy donors, ex vivo in IBD patients and in vivo in cynomolgus animal model of gastro-intestinal inflammation, and therefore can advantageously be used for treatment of gastro-intestinal inflammatory diseases such as IBD.

### SUMMARY

The present disclosure relates to an isolated anti-BTN3A antibody, for use in treating gastro-intestinal inflammatory disorders, such as inflammatory bowel disease, in a human subject in need thereof, wherein said anti-BTN3A antibody binds specifically to BTN3A1 and said anti-BTN3A antibody is selected among the anti-BTN3A antibodies which inhibit in vitro the degranulation of γδ T cells in co-culture with Daudi Burkitt's lymphoma cell lines with an IC50 of 10 nM or below, preferably 1 nM or below, for example as determined in a CD107 degranulation assay by flow cytometry.

The present disclosure also relates to methods of treating gastro-intestinal inflammatory disorders, such as inflammatory bowel disease, in a human subject in need thereof, said method comprising administering a therapeutically efficient amount of an isolated anti-BTN3A antibody to said subject, wherein said anti-BTN3A antibody binds specifically to BTN3A1 and said anti-BTN3A antibody is selected among the anti-BTN3A antibodies which inhibit in vitro the degranulation of γδ T cells in co-culture with Daudi Burkitt's lymphoma cell lines with an IC50 of 10 nM or below, preferably 1 nM or below, for example as determined in a CD107 degranulation assay by flow cytometry.

The present disclosure further relates to the use of an isolated anti-BTN3A antibody in a method for preparing a drug for treating gastro-intestinal inflammatory disorders, such as inflammatory bowel disease, in a human subject in need thereof, wherein said anti-BTN3A antibody binds specifically to BTN3A1 and said anti-BTN3A antibody is selected among the anti-BTN3A antibodies which inhibit in vitro the degranulation of γδ T cells in co-culture with Daudi Burkitt's lymphoma cell lines with an IC50 of 10 nM or below, preferably 1 nM or below, for example as determined in a CD107 degranulation assay by flow cytometry.

In specific embodiments of the above anti-BTN3A antibody and their methods of use, said antibody is selected from the group consisting of:
(i) an antibody mAb1 having a heavy chain of SEQ ID NO:21 and a light chain of SEQ ID NO:22,
(ii) a variant of mAb1 having a heavy chain variable region (VH) of SEQ ID NO:7, and, a light chain variable region (VL) of SEQ ID NO:8 but different constant regions,
(iii) a variant of mAb1 having HCDR1 of SEQ ID NO:1, HCDR2 of SEQ ID NO:2, HCDR3 of SEQ ID NO:3, LCDR1 of SEQ ID NO:4, LCDR2 of SEQ ID NO:5 and LCDR3 of SEQ ID NO:6 but different framework regions, or,
(iv) a variant of mAb1 which binds to the same epitope as mAb1, wherein said epitope comprises or essentially consists of SEQ ID NO:20, more preferably, it binds at least to residue 53, 62, 66 of BTN3A1, and wherein said variant does not have HCDR1 of SEQ ID NO:1, HCDR2 of SEQ ID NO:2, HCDR3 of SEQ ID NO:3, LCDR1 of SEQ ID NO:4, LCDR2 of SEQ ID NO:23 and LCDR3 of SEQ ID NO:6.

In more specific embodiments, the selected isolated anti-BTN3A antibody as disclosed herein, is a variant of mAb1 having HCDR1 of SEQ ID NO:1, HCDR2 of SEQ ID NO:2, HCDR3 of SEQ ID NO:3, LCDR1 of SEQ ID NO:4, LCDR2 of SEQ ID NO:5 and LCDR3 of SEQ ID NO:6, wherein the VH amino acid sequence has at least 90% identity but less than 100% identity with SEQ ID NO:7, preferably at least 95% identity, and the VL amino acid sequence has at least 90% identity but less than 100% identity with SEQ ID NO:8, preferably at least 95%.

In specific embodiments, the isolated anti-BTN3A antibody for use according to the present disclosure, binds to the human BTN3A1 isoform with a K_{D} of 10 nM or less, preferably with a K_{D} of 1 nM or less as measured by surface plasmon resonance, typically between 1.10⁻¹¹ and 1.10⁻⁹ M, as measured by surface plasmonic resonance (SPR) assay and/or binds to human peripheral blood mononuclear cells (PBMCs) with an EC50 of 0.1 μg/mL, or less, preferably with an EC50 of 0.05 μg/mL or less, for example between 0.1 μg/mL and 0.005 μg/mL, such as about 0.02 μg/mL.

In specific embodiments, the isolated anti-BTN3A antibody for use according to the present disclosure is a functional variant of mAb1 which retains at least a substantial proportion of the affinity of mAb1, preferably at least 90% of the affinity as measured by SPR assay, and has at least one or more of the following properties:
(i) it inhibits in vitro the degranulation of γδ T cells in co-culture with Daudi Burkitt's lymphoma cell lines with an EC50 of 10 nM or below, preferably 1 nM or below, for example as determined in a CD107 degranulation assay by flow cytometry;
(ii) it inhibits substantially the phosphoantigen mediated activation, gut homing potential, proliferation and degranulation capacity of peripheral Vδ2+ T cells from patients, for example as determined in an ex vivo assay with peripheral blood mononuclear cells (PBMCs) isolated from patients suffering from inflammatory bowel disease; and/or,
(iii) It inhibits substantially the phosphoantigen mediated activation and/or proliferation of gut Vδ2+ T cells from gut biopsies of inflammatory bowel disease patients, for example as determined by CD25 or HLA-DR expression in an ex vivo assay with walked out cells.

In specific embodiments, the isolated anti-BTN3A antibody for use according to the present disclosure, is a human or humanized antibody.

In specific embodiments, the isolated anti-BTN3A antibody for use according to the present disclosure includes an IgG Fc region, preferably a mutant or chemically modified IgG1 constant region wherein said mutant or chemically modified IgG1 constant region confers no or decreased binding to Fcγ receptors and/or ADCC mediating activity when compared to a corresponding antibody with wild type IgG1, for example a mutant IgG1 constant region having the following amino acid substitutions L247F L248E and P350S.

In specific embodiments, said gastric inflammatory disorder is inflammatory bowel disease, for example, ulcerative colitis or Crohn's disease.

Typically, the isolated anti-BTN3A antibody for use of the present disclosure as described herein, is administered intravenously to the subject at a dose of 1 to 100 mg.

In specific embodiments, the isolated anti-BTN3A antibody for use of the present disclosure is administered in combination, simultaneously or separately with an anti-inflammatory treatment, preferably selected from anti-cytokine antibodies (anti-IL-12, -IL-23, -TNFα), anti-a4b7 integrin antibodies, and JAK inhibitors.

### LEGENDS OF THE FIGURES

FIG. 1: a—epitope recognized by humanized selected variant of 103.2 clone; b—interaction BTN3A/clone 103.2. BTN3A PDB structure 4F80 was colored in dark grey on the epitope site. BTN3A amino acids colored in dark grey are corresponding to 53-66 (HLFPTMSAETMELK). A, B, C, D, E: ribbon/surface representation of front view (A); back view (B), side view 1 (C), side view 2 (D) and top view (E). F, G, H, I, J: ribbon representation of front view (F); back view (G), side view 1 (H), side view 2 (I) and top view (J).

FIG. 2: mAb1 inhibits activation phenotype, proliferation (CTV) and degranulation (CD107) abilities of Vδ2+ T cells from blood of IBD patients. (A) Graph on the left shows the frequency of Vδ2+ T cells in the blood of non IBD patients and IBD patients (n=9 for each group). The graphs on the right show the percentage of CD25+(top) or HLA-DR+(bottom) Vd2+ T cells assessed in the blood of non IBD patients (n=5 for CD25 and 6 for HLA-DR, grey bar) and IBD patients (n=7 for CD25 and 8 for HLA-DR, open bar) in response to cytokines (“IL-2/IL-15”), cytokines+phosphoantigens (“HDMAPP”), cytokines+phosphoantigens+isotype control (“hIgG1s”), cytokines+phosphoantigens+mAb1 (“mAb1”). Mean+/−SEM is represented. (B) Each graph represents the percentage of Vδ2 T cells positive for each marker assessed in the blood of non IBD patients (n=7, grey bar) and IBD patients (n=8, open bar) in response to cytokines (“IL-2/IL-15”), cytokines+phosphoantigens (“HDMAPP”), cytokines+phosphoantigens+isotype control (“hIgG1s”), cytokines+phosphoantigens+mAb1 (“mAb1”). Mean+/−SEM is represented.

FIG. 3: mAb1 inhibits activation phenotype, proliferation (CTV) and degranulation (CD107) abilities of Vδ2+ T cells from gut of IBD patients. Frequency of Vδ2+ T cells for the corresponding marker in the gut of non IBD patients (n=10 for CD25 and CTV, n=11 for HLA-DR and CD107, grey bar) and IBD patients (open bar) in response to cytokines (“IL-2/IL-15”), cytokines+phosphoantigens (“HDMAPP”), cytokines+phosphoantigens+isotype control (“hIgG1s”), cytokines+phosphoantigens+mAb1 (“mAb1”). Mean+/−SEM is represented.

FIG. 4: study design of the acute DSS-induced colitis cynomolgus model.

FIG. 5: iv administrations of mAb1 inhibit clinical signs associated to DSS-induced colitis cynomolgus monkeys. 7 animals per group were included in the study. Dotted lines indicate the days of dosing. *p<0.05, unpaired t test.

FIG. 6: iv administrations of mAb1 decrease circulating levels of pro-inflammatory cytokines IL-6 and IL-8 (FIG. 6A) and increase circulating levels of protective IL-17 and VEGF-A (FIG. 6B) in DSS-induced colitis cynomolgus monkeys. 7 animals per group were included in the study. Dotted lines indicate the days of dosing. Heatmaps (top panels) represent the fold change in the concentration quantified at the indicated timepoint versus baseline (d-3). Individual data per monkey are represented in the middle graphs. Mean concentration+/−standard error of the mean are represented for each group in the bottom panels.

### DETAILED DESCRIPTION

### Definitions

In order that the present disclosure may be more readily understood, certain terms are first defined. Additional definitions are set forth throughout the detailed description.

As used herein, the term “BTN3A” has its general meaning in the art and, unless otherwise specified, it refers to human BTN3A polypeptides including either BTN3A1 of SEQ ID NO:17, BTN3A2 of SEQ ID NO:18 or BTN3A3 of SEQ ID NO:19.

“Polypeptide,” “peptide” and “protein,” are used interchangeably and refer broadly to a polymer of amino acid residues of any length, regardless of modification (e.g., phosphorylation or glycosylation). The terms apply to amino acid polymers in which one or more amino acid residue is an artificial chemical mimetic of a corresponding naturally occurring amino acid, as well as to naturally occurring amino acid polymers and non-naturally occurring amino acid polymer. Polypeptides can be modified, e.g., by the addition of carbohydrate residues to form glycoproteins. The terms “polypeptide,” “peptide” and “protein” expressly include glycoproteins, as well as non-glycoproteins. In specific embodiments, the term “polypeptide” and “protein” refers to any polypeptide or protein which can be encoded by a gene and translated using cell expression system, such as mammalian host cell by recombinant means, including any polypeptide with post-translation modifications of the amino acid polymer or chemical modifications.

The term “recombinant protein”, as used herein, includes proteins that are prepared, expressed, created or isolated by recombinant means, such as (a) antibodies isolated from a hybridoma (described further below), (b) antibodies isolated from a production cell line transfected to express the corresponding heavy and light chains of said antibodies, e.g., from a transfectoma, etc.

The term “antibody” as used herein refers to immunoglobulin molecules and immunologically active portions of immunoglobulin molecules, i.e., molecules that contain an antigen binding site that specifically binds an antigen.

In natural antibodies of rodents and primates, two heavy chains are linked to each other by disulfide bonds, and each heavy chain is linked to a light chain by a disulfide bond. There are two types of light chains, lambda (1) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site, or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

The term “K_{assoc}” or “Kₐ”, as used herein, is intended to refer to the association rate of a particular antibody-antigen interaction, whereas the term “Kdis” or “K_{d},” as used herein, is intended to refer to the dissociation rate of a particular antibody-antigen interaction.

The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of a protein or an antibody is by using surface plasmon resonance, for example by using a biosensor system such as a Biacore® system. A simple binding interaction analysis by surface plasmon resonance (SPR) requires immobilization of the ligand to the sensor chip surface, followed by addition of the analyte of interest to the buffer flowing over the ligand surface. The interaction of the ligand and analyte is measured by the SPR instrument (typically the Biacore® system) as a change in refractive index over time. From this, the association (Kₐ) or dissociation (K_{d}) and equilibrium dissociation (K_{D}) constants can be derived.

The term “anti-BTN3A antibody” or “BTN3A antibody” as used herein refers to an antibody that has binding specificity to BTN3A.

As used herein, the term “binding specificity” refers to the ability of an antibody to detectably bind to an antigen recombinant polypeptide, such as recombinant BTN3A1 polypeptide, with a K_{D} of 100 nM or less, 10 nM or less, 1 nM or less, as measured by Surface Plasmon Resonance (SPR) measurements, for example as determined in the Examples (see Table 3). In some embodiments, the antibody binds to human BTN3A1 isoform with a K_{D} comprised between 10⁻³ pM and 100 nM, notably comprised between 10 pM and 100 nM, notably between 10 pM and 100 nM, or between 10⁻³ pM and 10 nM, notably 1 pM and 10 nM, notably between 10 pM and 10 nM, or between 1 pM and 5 nM, notably 10 pM and 5 nM or 100 pM and 5 nM as measured by SPR.

An antibody that “cross-reacts with an antigen other than BTN3A” is intended to refer to an antibody that binds that antigen other than human BTN3A with a K_{D} of 10 nM or less, 1 nM or less, or 100 pM or less. An antibody that “does not cross-react with a particular antigen” is intended to refer to an antibody that binds to that antigen, with a K_{D} of 100 nM or greater, or a K_{D} of 1 μM or greater, or a K_{D} of 10 μM or greater, said affinity being measured for example using similar Surface Plasmon Resonance (SPR) measurements as disclosed in the Examples. In certain embodiments, such antibodies that do not cross-react with the antigen exhibit essentially undetectable binding against these proteins in standard binding assays.

An anti-BTN3A antibody may have cross-reactivity to other antigens, such as related BTN3A molecules from other species, typically cynomolgus BTN3A1. Moreover, an isolated anti-BTN3A antibody may be substantially free of other cellular material and/or chemicals.

The phrases “an antibody recognizing an antigen” and “an antibody having specificity for an antigen” are used interchangeably herein with the term “an antibody which binds specifically to an antigen”.

Specificity can further be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a BTN3A polypeptide). The term “affinity”, as used herein, means the strength of the binding of an antibody to an epitope. Affinity is typically assessed by the K_{D} value of the antibody for BTN3A1.

“Humanized antibody” as used herein, refers broadly to include recombinant antibodies produced by a non-natural cell, for example a producing cell line, having variable and constant regions which have been altered to more closely resemble antibodies that would be made by a human cell. For example, by altering the non-human antibody amino acid sequence to incorporate amino acids found in human germline immunoglobulin sequences. The humanized antibodies for use of the disclosure may include amino acid residues not encoded by human germline immunoglobulin sequences (e.g., mutations introduced by random or site-specific mutagenesis in vitro or by somatic mutation in vivo), for example in the CDRs.

In specific embodiments, the term “humanized antibody”, as used herein, also includes antibodies in which CDR sequences derived from the germline of another mammalian species, such as a mouse, have been grafted onto human framework sequences.

In other specific embodiments, the term “humanized antibody”, as used herein, also includes antibodies in which H-CDR1 of SEQ ID NO:1, H-CDR2 of SEQ ID NO:2, H-CDR3 of SEQ ID NO:3, L-CDR1 of SEQ ID NO:4, L-CDR2 of SEQ ID NO:5, and L-CDR3 of SEQ ID NO:6 have been grafted onto human framework sequences.

As used herein, the term “inhibitory antibody” refers to an antibody able to directly or indirectly inhibits immune functions of effector cells, for example to inhibit proliferation and expansion, production of proinflammatory molecules, cytolytic functions against stressed cells, migration and trafficking properties and/or immunomodulatory responses including antigen presentation.

In preferred embodiments, an inhibitory BTN3A antibody has at least the capacity to inhibit in vitro the degranulation of γδ T cells (typically Vγ9Vδ2 T cells) in co-culture with cancer cells (such as Daudi Burkitt's lymphoma cell lines) (though other cells may be used that have been transformed or damaged), preferably with an IC₅₀ of 10 nM or below, more preferably of 1 nM or below, for example, 0.1 nM or below, as determined by flow cytometry in a CD107 degranulation assay, for example as described in the Examples below. IC₅₀ (half maximal inhibitory concentration) may be established in a dose response curve and represents the concentration of inhibitory BTN3A antibody where 50% of the maximal inhibitory effect (i.e.: the maximal inhibition of activated Vγ9Vδ2 T cells) is observed. As indicated in the Examples, assessment of the Vγ9Vδ2 T cells cytotoxicity is performed by evaluating the expression of CD107 molecule at their membrane surface by flow cytometry. Dose-response curves are typically established by quantifying CD107 positive Vγ9Vδ2 T cells after 4 h of co-culture with Daudi cells in presence of the antibody fragments at 37° C. In some embodiments, the IC₅₀ is comprised between 10⁻⁴ nM and 10 nM, notably between 10⁻⁴ nM and 1 nM, notably between 10⁻⁴ nM and 0.1 nM, or between 10⁻³ nM and 10 nM, 10⁻³ nM and 1 nM or between 10⁻³ nM and 0.1 nM.

As used herein, the term “subject” includes any human or non-human animal. The term “non-human animal” includes all vertebrates, e.g., mammals and non-mammals, such as non-human primates, sheep, dogs, cats, horses, cows, chickens, amphibians, reptiles, etc.

As used herein, the percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the Needleman and Wunsch algorithm (NEEDLEMAN, and Wunsch).

The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification.

### Inhibitory BTN3A Antibodies for Use According to the Present Disclosure

The present disclosure relates to inhibitory BTN3A antibody, for use in treating gastro-intestinal inflammatory disorders, such as IBDs in a human subject in need thereof, wherein said inhibitory BTN3A antibody binds specifically to BTN3A and inhibits the cytolytic function of γδ T cells as determined in an in vitro degranulation assay; e.g. the CD107 degranulation assay in the presence of Daudi cells as described in the Examples.

In specific embodiments, inhibitory BTN3A antibody binds to the human BTN3A1 isoform with a K_{D} of 10 nM or less, preferably with a K_{D} of 1 nM or less as measured by surface plasmon resonance, typically between 1.10⁻¹¹ and 1.10⁻⁹ M, as measured by surface plasmonic resonance (SPR) assay and/or which binds to human peripheral blood mononuclear cells (PBMCs) with an EC50 of 0.1 μg/mL, or less, preferably with an EC50 of 0.05 μg/mL or less, for example about 0.02 μg/mL.

Preferably, the inhibitory BTN3A antibody for use of the present disclosure is a chimeric, humanized or human antibody. Typically, a non-human antibody is humanized to reduce immunogenicity to humans, while having at least the same or similar affinity (or superior affinity) of the parental non-human antibody.

Generally, a humanized antibody comprises one or more variable domains in which, CDRs, (or portions thereof) are derived from a non-human antibody, e.g. the murine mAb 103.2, optionally with one amino acid substitution in one CDR to reduce immunogenicity, in particular in L-CDR2, such as L-CDR2 of SEQ ID NO:5 wherein an isoleucine of the murine L-CDR2 of SEQ ID NO:24 has been replaced by an alanine, and FRs (or portions thereof) are derived from the murine antibody sequences with mutations to reduce immunogenicity.

A humanized antibody for use according to the present disclosure optionally will also comprise at least a portion or all of a human constant region.

In specific embodiments, the inhibitory BTN3A antibody for use according to the present disclosure includes an IgG Fc region, preferably a mutant or chemically modified IgG1 or IgG4 constant region wherein said mutant or chemically modified IgG1 or IgG4 constant region confers no or decreased binding to Fcγ receptors and/or ADCC mediating activity when compared to a corresponding antibody with wild type IgG1 or IgG4 isotype constant region, for example a mutant IgG1 constant region having the following amino acid substitutions: L247F L248E P350S.

In specific embodiments, the inhibitory BTN3A antibody is further selected among BTN3A antibody which inhibits phosphoantigen mediated activation, gut homing potential, proliferation and degranulation capacity of peripheral Vδ2+ T cells, for example as measured in an ex vivo assay with PBMC isolated from patients suffering from IBDs. The protocol of such ex vivo assay is described in more details in the Examples. Briefly, inhibition of phosphoantigen mediated ex vivo activation of peripheral Vδ2+ T cells may be determined by an ex vivo assay consisting for example of culturing PBMCs isolated from blood or gut biopsies of Crohn's Disease or Ulcerative Colitis patients and stained with 1 μM CTV dye, for 4 days in the presence of (i) IL2 (20 IU/ml)/IL5 (20 ng/ml), (ii) 1 nM to 10 nM HDMAPP (phosphoantigen) and (iii) 1 μg/mL of said inhibitory BTN3A or a control isotype. Activation may be determined by measuring several markers such as CD25, HLA-DR, b7 integrin, or CD49b. Proliferation may be determined by flow cytometry by measuring CTV dilution, and degranulation may be determined by determining CD107a expression by flow cytometry, for Vδ2+ and Vδ2− T cells.

In specific embodiments, the inhibitory BTN3A antibody is further selected among BTN3A antibodies which inhibit substantially the phosphoantigen mediated activation and/or proliferation of gut Vδ2+ T cells from gut biopsies of IBD patients, for example as determined by CD25 or HLA-DR expression in the ex vivo assay with walked out cells as described in the Examples below.

In preferred embodiments, said inhibitory BTN3A antibody also cross-reacts with cynomolgus BTN3A.

### Reference Antibodies for Use in Treating Gastro-Intestinal Inflammatory Disorders

In preferred embodiments, said inhibitory BTN3A antibody for use in treating gastro-intestinal inflammatory disorders such as IBDs in a human subject in need thereof, is mAb1 antibody having a full-length heavy chain of SEQ ID NO:21, and a full-length light chain of SEQ ID NO:22.

In other specific embodiments, said inhibitory BTN3A antibody for use in treating gastro-intestinal inflammatory disorders such as IBDs in a human subject in need thereof, is a variant of mAb1 antibody having a heavy chain variable region (VH) of SEQ ID NO:7, and, a light chain variable region (VL) of SEQ ID NO:8 but different constant region. In this embodiment, the variant of mAb1 has variant constant region as compared to mAb1, for example, a different isotype or the same isotype but with one or more amino acid mutations as compared to mAb1 corresponding Fc region.

As used herein, a variant antibody is an antibody which has distinct primary amino acid sequence as compared to the reference antibody mAb1, for example by one or more amino acid additions, deletions, and/or substitutions. Preferably a variant antibody is an antibody with distinct primary amino acid sequence of the heavy or light chain by one or more amino acid substitutions, for example 1, 2, 3, 4, 5, 6, 7, 8, 9 or 10 amino acid substitutions in the heavy and/or light chain of mAb1.

In specific embodiments, the variant of mAb1 has variant constant regions as compared to mAb1, for example, a different isotype from mAb1 or the same isotype but with one or more amino acid mutations as compared to mAb1, for example 1, 2, 3, 4, 5, 6, 7, 8, 9 or 10 amino acid substitutions in the Fc region as compared to mAb1.

In other specific embodiments, said inhibitory BTN3A antibody for use in treating gastro-intestinal inflammatory disorders such as IBDs in a human subject, is a variant of mAb1 having HCDR1 of SEQ ID NO:1, HCDR2 of SEQ ID NO:2, HCDR3 of SEQ ID NO:3, LCDR1 of SEQ ID NO:4, LCDR2 of SEQ ID NO:5 and LCDR3 of SEQ ID NO:6 but different framework regions as compared to mAb1.

The CDR regions of the inhibitory BTN3A antibody of the present disclosure are delineated using the Kabat numbering (Kabat et al., 1992, hereafter “Kabat et al.”). For the ease of reading, H-CDR1, H-CDR2, H-CDR3 refer to the 3 CDRs of the VH region and L-CDR1, L-CDR2, L-CDR3 refer to the 3 CDRs of the VL region.

In other specific embodiments, said inhibitory BTN3A antibody for use according to the present disclosure, is a variant of mAb1 antibody having HCDR1 of SEQ ID NO:1, HCDR2 of SEQ ID NO:2, HCDR3 of SEQ ID NO:3, LCDR1 of SEQ ID NO:4, LCDR2 of SEQ ID NO:5 and LCDR3 of SEQ ID NO:6, wherein the VH region has at least 90% identity, preferably at least 95% identity with SEQ ID NO:7, and the VL region has at least 90% identity preferably at least 95% with SEQ ID NO:8.

Preferably, said inhibitory BTN3A antibody is a humanized BTN3A antibody comprising:
(i) a heavy chain of an IgG antibody with VH of SEQ ID NO:7, and,
(ii) a light chain of an IgG antibody with VL of SEQ ID NO:8.

In certain embodiments, an inhibitory BTN3A antibody according to the disclosure having VH and VL sequences can be used to create new BTN3A antibodies respectively, by modifying the VH and/or VL sequences, or the constant regions attached thereto.

Thus, in another aspect according to at least some embodiments of the disclosure, the structural features of an inhibitory BTN3A antibody, are used to create structurally related BTN3A antibodies that retain at least the inhibitory property of the reference inhibitory BTN3A antibody mAb1.

For example, the 6 CDR regions of mAb1 or an antibody with H-CDR1 of SEQ ID NO:1, H-CDR-2 of SEQ ID NO:2, and H-CDR3 of SEQ ID NO:3; L-CDR1 of SEQ ID NO:4, L-CDR2 of SEQ ID NO:5, and a L-CDR3 of SEQ ID NO:6 can be combined recombinantly with known framework regions to create additional, recombinantly-engineered, inhibitory BTN3A antibodies according to at least some embodiments of the disclosure, as discussed above. The starting material for the engineering method is one or more of the VH and/or VL sequences with SEQ ID NO:7 and SEQ ID NO:8.

The functional properties of the altered antibodies can be assessed using standard assays available in the art and/or described herein. In certain embodiments of the methods of engineering antibodies according to at least some embodiments of the disclosure, mutations can be introduced randomly or selectively along all or part of inhibitory BTN3A antibody coding sequence and the resulting modified inhibitory BTN3A antibody can be screened for binding activity and/or other desired functional properties, such as in vitro inhibitory property in a degranulation assay, or inhibition of ex vivo activation, gut homing potential, proliferation and degranulation capacity of peripheral Vδ2+ T cells isolated from patients suffering from IBDs (as described in the previous sections).

Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### Isotype and Fc Engineering

In preferred embodiments, an inhibitory BTN3A antibody for use according to the present disclosure includes the constant region of an immunoglobulin, preferably an IgG antibody.

As used herein, the term “constant region” or “Fc region” is used interchangeably to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody wherein the numbering is according to the EU numbering system. The C-terminal lysine (residue K447) of the Fc region may be removed, for example, during production or purification of the antibody or its corresponding codon deleted in the recombinant constructs. Accordingly, a composition of antibodies of the disclosure may comprise antibody populations with all K447 residues removed, antibody populations with no K447 residues removed, and antibody populations having a mixture of antibodies with and without the K447 residue.

The constant region of an inhibitory BTN3A antibody for use according to the present disclosure may be of any isotype. The choice of isotype typically will be guided by the desired effector functions, such as ADCC silencing. Exemplary isotypes are IgG1, IgG2, IgG3, and IgG4. Either of the human light chain constant regions, kappa or lambda, may be used. If desired, the class of an antibody of the present disclosure may be switched by known methods. Typical, class switching techniques may be used to convert one IgG subclass to another, for instance from IgG1 to IgG2. Thus, the effector function of the antibodies of the present disclosure may be changed by isotype switching to, e.g., an IgG1, IgG2, IgG3, IgG4, IgD, IgA, IgE, or IgM antibody for various therapeutic uses. In some embodiments, the antibody of the disclosure is a full-length antibody. In some embodiments, the full-length antibody is an IgG1 antibody.

The inhibitory BTN3A antibody for use according to the present disclosure may be engineered to include modifications within the Fc region, typically to alter one or more functional properties of the antibody, such as serum half-life, complement fixation, Fc receptor binding, and/or antigen-dependent cellular cytotoxicity, more specifically modification of the Fc region of IgG1 isotype, and typically by amino acid substitutions.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In other embodiments, the Fc region is modified to decrease the ability of the antibody to mediate antibody dependent cellular cytotoxicity (ADCC) and/or to decrease the affinity of the antibody for an Fcγ receptor by modifying one or more amino acids. Such antibodies with decreased effector functions, and in particular decreased ADCC include silent antibodies.

In certain embodiments, the Fc domain of the IgG1 isotype is used. In some specific embodiments, a mutant variant of the IgG1 Fc fragment is used, e.g. a silent IgG1 Fc which reduces or eliminates the ability of the fusion polypeptide to mediate antibody dependent cellular cytotoxicity (ADCC) and/or to bind to an Fcγ receptor.

In certain embodiments, the Fc domain of the IgG4 isotype is used. In some specific embodiments, a mutant variant of the IgG4 Fc fragment is used, e.g. a silent IgG4 Fc which reduces or eliminates the ability of the fusion polypeptide to mediate antibody dependent cellular cytotoxicity (ADCC) and/or to bind to an Fcγ receptor.

Silenced effector functions can be obtained by mutation in the Fc constant part of the antibodies and have been described in the Art (Baudino et al., 2008; Strohl, 2009). Examples of silent IgG1 antibodies comprise the triple mutant variant IgG1 L247F L248E P350S. Examples of silent IgG4 antibodies comprise the double mutant variant IgG4 S241P L248E.

In other specific embodiments, the antibodies for use of the present disclosure includes constant regions which are not selected among the double mutant variant IgG4 S241P L248E and/or the triple mutant variant IgG1 L247F L248E P350S.

In certain embodiments, the Fc domain is a silent Fc mutant preventing glycosylation at position 314 of the Fc domain. For example, the Fc domain contains an amino acid substitution of asparagine at position 314. An example of such amino acid substitution is the replacement of N314 by a glycine or an alanine.

In some embodiments, the full-length inhibitory BTN3A1 antibody of the disclosure is an IgG1 antibody. In some embodiments, the full-length inhibitory BTN3A1 antibody of the disclosure is an IgG1 antibody having the triple mutations L247F L248E P350S.

Furthermore, inhibitory BTN3A antibody for use according to the present disclosure may be chemically modified (e.g., one or more chemical moieties can be attached to the antibody) or may be modified to alter its glycosylation, again to alter one or more functional properties of the antibody. Each of these embodiments is described in further detail below.

A modification of the antibodies herein that is contemplated by the disclosure is pegylation or hesylation or related technologies. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In some embodiments, the antibody to be pegylated is an aglycosylated antibody. Methods for pegylating proteins are known in the art and can be applied to the antibodies of the present disclosure. See for example, EP 0154 316 by Nishimura et al. and EP 0 401 384 by Ishikawa et al.

Another modification of the antibodies that is contemplated by the disclosure is a conjugate or a protein fusion of at least the antigen-binding region of the antibody of the present disclosure to serum protein, such as human serum albumin or a fragment thereof to increase half-life of the resulting molecule.

### Functional Variant Antibodies

In yet another embodiment, a functional variant antibody of the disclosure has full length heavy and light chain amino acid sequences; or variable region heavy and light chain amino acid sequences, or all 6 CDR regions amino acid sequences that are homologous or more specifically identical to the corresponding amino acid sequences of the reference antibody mAb1 as described above, and wherein such functional variant antibodies exhibit the desired functional properties of said reference antibody mAb1, preferably to substantially the same level.

A functional variant of the reference mAb1 antibody, notably a functional variant of a VL, VH, or CDR used in the context of the present disclosure still allows the antibody to retain at least a substantial proportion (at least about 50%, 60%, 70%, 80%, 90%, 95% or 100%) of the affinity (typically assessed by K_{D} as measured by SPR assay, and/or the specificity/selectivity of the parent antibody (e.g.: mAb1)) and in some cases such a monoclonal antibody of the present disclosure may be associated with greater affinity, selectivity and/or specificity than the parent Ab (e.g.: mAb1).

Desired functional properties of the reference mAb1 may be selected from one or more (preferably all) of the following properties:
(i) binding to the human BTN3A1 isoform, preferably with a K_{D} of 10 nM or less, preferably with a K_{D} of 1 nM or less as measured by surface plasmon resonance, typically between 1.10⁻¹¹ and 1.10⁻⁹ M, as measured by SPR assay; for example as determined in the Biacore assay as described in the Examples below;
(ii) binding to human PBMCs, preferably with an EC50 of 0.1 μg/mL, or less, preferably with an EC50 of 0.05 μg/mL or less, for example about 0.02 μg/mL; for example between 0.1 μg/mL and 0.005 μg/mL as determined in the PBMC binding assay as described in the Examples below;
(iii) inhibiting in vitro the degranulation of γδ T cells (typically Vγ9Vδ2 T cells) in co-culture with cancer cells such as Daudi Burkitt's lymphoma cell lines, preferably with an IC₅₀ of 10 nM or below, preferably of 1 nM or below, for example, 0.1 nM or below, for example as determined in a CD107 degranulation assay as described in the Examples below;
(iv) inhibiting substantially the phosphoantigen mediated activation, gut homing potential, proliferation and degranulation capacity of peripheral Vδ2+ T cells from patients, for example as determined in an ex vivo assay with PBMC isolated from IBD patients as described in the Examples below and/or
(v) inhibiting substantially the phosphoantigen mediated activation and/or proliferation of gut Vδ2+ T cells from gut biopsies of IBD patients, for example as determined by CD25 or HLA-DR expression in the ex vivo assay with walked out cells as described in the Examples below.

In specific embodiments, the isolated anti-BTN3A antibody for use according to the present disclosure is a functional variant of mAb1 which retains at least a substantial proportion of the affinity of mAb1, preferably at least 90% of the affinity as measured by SPR assay, and has at least one or more of the following properties:
(i) it inhibits in vitro the degranulation of γδ T cells in co-culture with Daudi Burkitt's lymphoma cell lines with an IC50 of 10 nM or below, preferably 1 nM or below, for example as determined in a CD107 degranulation assay by flow cytometry;
(ii) it inhibits substantially the phosphoantigen mediated activation, gut homing potential, proliferation and degranulation capacity of peripheral Vδ2+ T cells from patients, for example as determined in an ex vivo assay with PBMC isolated from patients suffering from IBDs, and/or;
(iii) it inhibits substantially the phosphoantigen mediated activation and/or proliferation of gut Vd2+ T cells from gut biopsies of IBD patients, for example as determined by CD25 or HLA-DR expression in the ex vivo assay with walked out cells as described in the Examples below.

For example, the disclosure relates to functional variant antibodies of mAb1, comprising a variable heavy chain (V_{H}) and a variable light chain (V_{L}) sequences where the CDR sequences, i.e. the 6 CDR regions; HCDR1, HCDR2, HCDR3, LCDR1, LCDR2, LCDR3 share all at least 80, 90 or 100 percent sequence identity to the corresponding CDR sequences of mAb1, wherein said functional variant antibody specifically binds to BTN3A1, and the antibody exhibits at least one of the following functional properties:
(i) It binds to the human BTN3A1 isoform with a K_{D} of 10 nM or less, preferably with a K_{D} of 1 nM or less, typically between 1.10⁻¹¹ and 1.10⁻⁹ M, as measured by SPR assay; for example as determined in the Biacore assay as described in the Examples below;
(ii) It binds to human PBMCs with an EC50 of 0.1 μg/mL, or less, preferably with an EC50 of 0.05 μg/mL or less, for example about 0.02 μg/mL; for example as determined in the PBMC binding assay as described in the Examples below;
(iii) it inhibits in vitro the degranulation of γδ T cells in co-culture with Daudi Burkitt's lymphoma cell lines with an IC50 of 10 nM or below, preferably 1 nM or below, for example as determined in a CD107 degranulation assay by flow cytometry;
(iv) it inhibits substantially the phosphoantigen mediated activation, gut homing potential, proliferation and degranulation capacity of peripheral Vδ2+ T cells from patients, for example as determined in an ex vivo assay with PBMC isolated from patients suffering from IBDs;
(v) it inhibits substantially the phosphoantigen mediated activation and/or proliferation of gut Vδ2+ T cells from gut biopsies of IBD patients, for example as determined by CD25 or HLA-DR expression in the ex vivo assay with walked out cells as described in the Examples below; and/or,
(vi) It cross-reacts with cynomolgus BTN3A1.

It further relates to functional variant antibodies of mAb1, comprising a heavy chain variable region and a light chain variable region that are at least 80%, 90%, or at least 95% or 100% identical to the corresponding heavy and light chain variable regions of mAb1; the functional variant antibody specifically binds to BTN3A1, and exhibits at least one (preferably all) of the following functional properties:
(i) It binds to the human BTN3A1 isoform with a K_{D} of 10 nM or less, preferably with a K_{D} of 1 nM or less, typically between 1.10⁻¹¹ and 1.10⁻⁹ M, as measured by SPR assay; for example as determined in the Biacore assay as described in the Examples below;
(ii) It binds to human PBMCs with an EC50 of 0.1 μg/mL, or less, preferably with an EC50 of 0.05 μg/mL or less, for example about 0.02 μg/mL; for example as determined in the PBMC binding assay as described in the Examples below;
(iii) it inhibits in vitro the degranulation of γδ T cells in co-culture with Daudi Burkitt's lymphoma cell lines with an IC50 of 10 nM or below, preferably 1 nM or below, for example as determined in a CD107 degranulation assay by flow cytometry;
(iv) it inhibits substantially the phosphoantigen mediated activation, gut homing potential, proliferation and degranulation capacity of peripheral Vδ2+ T cells from patients, for example as determined in an ex vivo assay with PBMC isolated from patients suffering from IBDs;
(v) it inhibits substantially the phosphoantigen mediated activation and/or proliferation of gut Vδ2+ T cells from gut biopsies of IBD patients, for example as determined by CD25 or HLA-DR expression in the ex vivo assay with walked out cells as described in the Examples below; and/or,
(vi) It cross-reacts with cynomolgus BTN3A1.

In certain embodiments, the sequences of CDR functional variants may differ from the sequence of the CDRs of the parent/reference antibody sequences of mAb1 through mostly conservative substitutions; for instance at least 10, such as at least 9, 8, 7, 6, 5, 4, 3, 2 or 1 of the substitutions in the variant are conservative amino acid residue replacements. In the context of the present disclosure, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also is substantially retained in a variant CDR as compared to a CDR of the mAb1.

The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=I I and Extended Gap=I).

Suitable variants typically exhibit at least about 90%, for example 95% of identity to the parent polypeptide VH and VL sequences. According to the present disclosure a first amino acid sequence having at least 90% of identity with a second amino acid sequence means that the first sequence has 90; 91; 92; 93; 94; 95; 96; 97; 98; 99; or 100% of identity with the second amino acid sequence. According to the present disclosure a first amino acid sequence having at least 50% of identity with a second amino acid sequence means that the first sequence has 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with the second amino acid sequence.

In some embodiments, the functional variant is a humanized antibody. In specific embodiments, the antibody of the present disclosure is a humanized antibody which comprises the 6 CDRs of mAb1 reference antibodies and alternative humanized framework region as compared to the humanized framework regions of mAb1.

Functional variant antibodies with mutant amino acid sequences can be obtained by mutagenesis (e.g., site-directed or PCR-mediated mutagenesis) of the coding nucleic acid molecules, followed by testing of the encoded altered antibody for retained function (i.e., the functions set forth above) using the functional assays described herein.

Antibodies that Cross-Compete at Least with mAb1 and/or that Bind to the Same Epitope as mAb1

Additional antibodies with similar advantageous properties of the reference antibody mAb1 as disclosed herein can be identified based on their ability to cross-compete (e.g., to competitively inhibit the binding of), or to bind to the same epitope as mAb1, in a statistically significant manner with mAb1 as described above, in standard BTN3A1 binding assays.

Test antibody may first be screened for their binding affinity to BTN3A1, for example from human recombinant antibody libraries using for example phage display technologies or from transgenic mouse expressing human variable region antibodies immunized with BTN3A1 antigens.

The ability of a test antibody to cross-compete with or inhibit the binding of antibodies of the present disclosure to human BTN3A1 demonstrates that the test antibody can compete with that antibody for binding to human BTN3A1; such an antibody may, according to non-limiting theory, bind to the same or a related (e.g., a structurally similar or spatially proximal) epitope on human BTN3A1 as the antibody with which it competes.

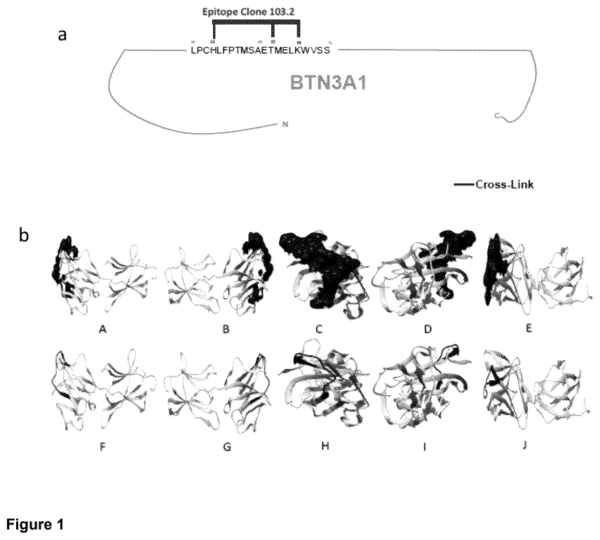

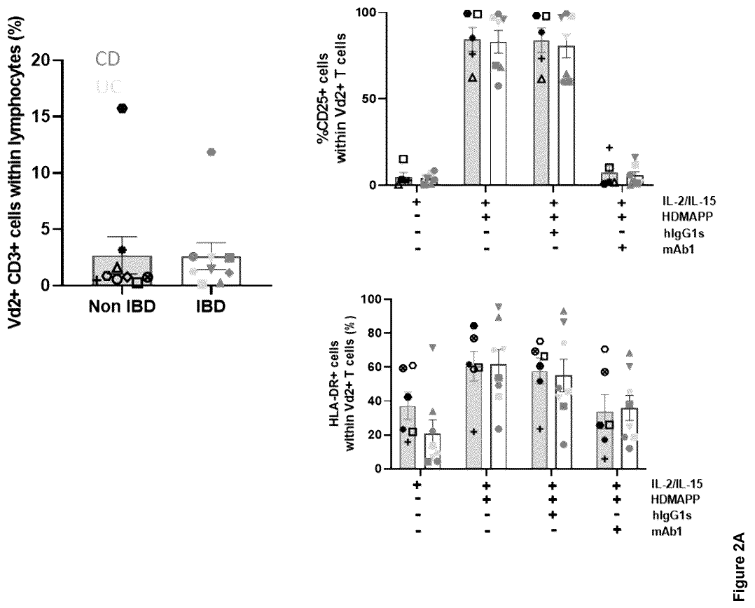

The epitope of mAb1 has been determined and it has been shown that mAb1 binds at least to residues 53, 62 and 66 of BTN3A1 (see for example FIG. 1) and therefore the epitope includes or essentially consists of SEQ ID NO:20 of BTN3A1.

In specific embodiments, the disclosure provides inhibitory BTN3A antibodies that bind to the same epitope as do at least the reference inhibitory BTN3A1 antibody mAb1 as described herein.

To screen an anti-BTN3A1 antibody for its ability to bind to the same epitope as one of mAb1 reference antibodies, for example, BTN3A-KO HEK293 cells transfected with human BTN3A1 are stained with saturating concentration (10 μg/mL) of one of the reference antibodies mAb1 during 30 minutes at 4° C. After 2 washes, different doses of a test anti-BTN3A1 mAbs are tested (30 minutes at 4° C.) for their competitive potential with any one of mAb1 reference antibodies. The mAbs that do compete for the same binding site as the reference antibody will not be able to recognize BTN3A1 in the presence of such reference antibodies. The data can be expressed as mean fluorescence intensity. Alternatively, competition assay can be performed in a binning experiment using biolayer interferometry (BLI) by immobilizing recombinant human BTN3A1 on a Biosensor and by adding the reference antibody followed by the potentially competing antibody.

The selected antibodies can be further tested for the advantageous properties of mAb1 in particular with respect to inhibitory properties against phosphoantigen mediated activation of Vγ2 T cells.

Accordingly, in one embodiment, the disclosure provides an isolated antibody which competes for binding of mAb1 to BTN3A1 or which binds to the same epitope of mAb1 (typically including the amino acids 52, 62, and 66 of BTN3A1), wherein said antibody exhibits at least one of the following properties:
(i) it binds to the human BTN3A1 isoform with a K_{D} of 10 nM or less, preferably with a K_{D} of 1 nM or less, typically between 1.10⁻¹¹ and 1.10⁻⁹ M, as measured by SPR assay; for example as determined in the Biacore assay as described in the Examples below;
(ii) it binds to human PBMCs with an EC50 of 0.1 μg/mL, or less, preferably with an EC50 of 0.05 μg/mL or less, for example about 0.02 μg/mL; for example as determined in the PBMC binding assay as described in the Examples below;
(iii) it inhibits in vitro the degranulation of γδ T cells in co-culture with Daudi Burkitt's lymphoma cell lines with an IC50 of 10 nM or below, preferably 1 nM or below, for example as determined in a CD107 degranulation assay by flow cytometry;
(iv) it inhibits substantially the phosphoantigen mediated activation, gut homing potential, proliferation and degranulation capacity of peripheral Vδ2+ T cells from patients, for example as determined in an ex vivo assay with PBMC isolated from patients suffering from IBDs; and/or
(v) it inhibits substantially the phosphoantigen mediated activation and/or proliferation of gut Vd2+ T cells from gut biopsies of IBD patients, for example as determined by CD25 or HLA-DR expression in the ex vivo assay with walked out cells as described in the Examples below.

Typically, functional properties according to points (iii), (iv) and/or (v) above of an antibody that competes with mAb1 for binding to BTN3A1 or binds to the same epitope as mAb1, are substantially equal or superior to the corresponding functional properties of mAb1, as described above. By substantially equal it is herein intended that the functional variant retains at least about 50%, 60%, 70%, 80%, 90%, 95% or 100% of the corresponding functional property of said reference mAb1.

Typically, an antibody that competes with mAb1 for binding to BTN3A1 or binds to the same epitope as mAb1 according to the present disclosure, still has at least a substantial proportion (at least about 50%, 60%, 70%, 80%, 90%, 95% or 100%) of the affinity of the reference antibody and in some cases may be associated with greater affinity, selectivity and/or specificity than the reference antibody mAb1.

In a specific embodiment, the disclosure provides antibodies, preferably chimeric, humanized or human recombinant antibodies, that bind or cross-compete to the same epitope as the inhibitory BTN3A antibody mAb1.

### Pharmaceutical Compositions

In another aspect, the present disclosure provides a composition, e.g., a pharmaceutical composition, containing an inhibitory BTN3A antibody as disclosed above, formulated together with a pharmaceutically acceptable carrier.

In another aspect, the present disclosure provides a composition, e.g., a pharmaceutical composition, containing one or a combination of antibodies disclosed herein, for example, mAb1 or their variants, or antigen-binding fragments, formulated together with a pharmaceutically acceptable carrier. Such compositions may include one or a combination of (e.g., two or more different) antibodies as described above.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). In one embodiment, the carrier should be suitable for subcutaneous route or intravenous injection.

Sterile phosphate-buffered saline is one example of a pharmaceutically acceptable carrier. Other suitable carriers are well-known to those in the art. Compositions comprising such carriers are formulated by well-known conventional methods (see, for example, Remington's Pharmaceutical Sciences, 18th edition, A. Gennaro, ed., Mack Publishing Co., Easton, PA, 1990; and Remington, The Science and Practice of Pharmacy, 20th Ed., Mack Publishing, 2000)). Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for a topical, oral, parenteral, intranasal, intravenous, intramuscular, subcutaneous or intraocular administration and the like.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the antibody may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders or lyophilisates for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose.

Dispersions can also be prepared in glycerol, liquid polyethylene glycols, and mixtures thereof and in oils. Under ordinary conditions of storage and use, these preparations contain a preservative to prevent the growth of microorganisms.

An antibody of the disclosure can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small inflamed tissue area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibodies of the disclosure may be formulated within a therapeutic mixture to comprise about 1 to 100 mg per dose. Multiple doses can also be administered.

Suitable formulation for solution for infusion or subcutaneous injection of antibodies have been described in the art and for example are reviewed in Cui et al (Drug Dev Ind Pharm 2017, 43(4): 519-530).

In certain embodiments, the use of liposomes and/or nanoparticles is contemplated for the introduction of antibodies into host cells. The formation and use of liposomes and/or nanoparticles are known to those of skill in the art.

### Uses and Methods of the BTN3A Inhibitory Antibodies of the Disclosure

The inhibitory BTN3A antibody of the present disclosure has in vitro and in vivo diagnostic and therapeutic utilities.

The above disclosed inhibitory BTN3A antibodies are indeed useful in methods for preparing a medicament for use in treating gastro-intestinal inflammation, such as IBDs in a human subject in need thereof.

In particular, the inhibitory BTN3A antibody can inhibit in vivo the cytolytic function, cytokine production and/or the proliferation of Vδ2 T cells mediated by phosphoantigens, as suggested in vitro or ex vivo using isolated immune cells from IBD patients.

Therefore, the inhibitory BTN3A antibodies of the disclosure may be used in methods for inhibiting the activation of Vδ2 T cells in a subject in need thereof, especially, in terms of migration, antigen presentation, cytokine secretion or cytolytic function, said methods comprising administering to the subject in need thereof, an inhibitory effective amount of said inhibitory BTN3A antibody of the present disclosure, e.g, mAb1 as disclosed herein.

Another object of the present disclosure thus relates to a method of inhibiting an immune response in a subject in need thereof, in particular inhibiting the cytolytic property of activated Vδ2 T cells in a subject in need thereof, comprising administering to the subject a therapeutically effective amount of said inhibitory BTN3A antibody of the present disclosure, e.g., mAb1 as disclosed herein.

In specific embodiments, a subject in need thereof is selected among the subjects suffering from gastro-intestinal disorders, e.g., IBD, and exhibiting higher blood cell counts of activated 62 T cells prior to treatment with said inhibitory BTN3A antibodies, as compared to a control. Such control can be for example average blood cell counts of activated 62 T cells in healthy subjects.

In other specific embodiments, a subject in need thereof is selected among the subjects suffering from gastro-intestinal disorders, e.g., IBD, based on BTN3A expression level in the gut, typically exhibiting a high level of BTN3A expression in the gut, as compared to a control. Such control can be for example average BNT3A expression in healthy subjects.

In specific embodiments, the inhibitory BTN3A antibodies of the disclosure are useful in particular for treating, preventing or diagnosing gastro-intestinal inflammatory disorders such as IBDs, in particular gastro-intestinal inflammatory disorders where activated 62 T cells are involved, for example ulcerative colitis or Crohn's disease.

Other gastro-intestinal inflammatory disorders where activated 62 T cells are involved which can be advantageously treated with the inhibitory BTN3A antibodies include without limitation: peptic ulcer disease, gastritis, gastroenteritis, celiac disease, appendicitis, pancreatitis, Whipple's disease, hepatitis, enteritis, enterocolitis, duodenitis, jejunitis and ileitis, cholangitis.

The disclosure also pertains to the methods of manufacturing a medicament for use in the treatment of gastro-intestinal inflammatory disorders such as IBDs, for example ulcerative colitis or Crohn's disease, said medicament comprising an inhibitory BTN3A antibody of the present disclosure, as described in the previous sections, for example mAb1 as disclosed herein.

The antibodies of the disclosure may be administered as the sole active ingredient or in conjunction with, e.g. as an adjuvant to or in combination (concomitantly or sequentially) to, other drugs e.g. immunosuppressive or immunomodulating agents or other anti-inflammatory agents, e.g. for the treatment or prevention of diseases mentioned above.

Typically, said anti-inflammatory agent may include without limitation, an anti-inflammatory cytokine, said cytokine being optionally selected from interleukin (IL)-12, IL-22, IL-23, Tumor Necrosis Factor (TNF) alpha. In other embodiment, said anti-inflammatory agent may include without limitation a steroid, for example a glucocorticoid, prednisone, hydrocortisone, 5-ASA, cyclosporine A (CsA) or an immunomodulator including an anti-a4 integrin, an anti-a4b7 integrin, a JAK inhibitor, azathioprine, mercaptopurine, or methotrexate.

As used herein, the term “treatment” or “treat” refer to both prophylactic or preventive treatment as well as curative or disease modifying treatment, including treatment of subject at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

As used herein, the term “therapeutically effective amount” refers to an amount effective, at dosages and for periods of time necessary, to achieve a desired therapeutic result. A therapeutically effective amount of the antibody of the present disclosure may vary according to factors such as the disease state, age, sex, and weight of the individual, and the ability of the antibody of the present disclosure to elicit a desired response in the individual. A therapeutically effective amount is also one in which any toxic or detrimental effects of the antibody or antibody portion are outweighed by the therapeutically beneficial effects. The efficient dosages and dosage regimens for the antibody of the present disclosure depend on the disease or condition to be treated and may be determined by the persons skilled in the art. A physician having ordinary skill in the art may readily determine and prescribe the effective amount of the pharmaceutical composition required. For example, the physician could start doses of the antibody of the present disclosure employed in the pharmaceutical composition at levels lower than that required in order to achieve the desired therapeutic effect and gradually increase the dosage until the desired effect is achieved. In general, a suitable dose of a composition of the present disclosure will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to treat inflammatory disorders, for example, be evaluated in an animal model system predictive of efficacy in treating inflammatory disorders. Alternatively, this property of a composition may be evaluated by examining the ability of the compound to inhibit induction of immune response by in vitro assays known to the skilled practitioner. A therapeutically effective amount of a therapeutic compound may decrease immune or inflammatory response, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of an antibody of the present disclosure is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present disclosure is 0.01-100 mg/kg, such as about 0.01-30 mg/kg, such as about 0.05-10 mg/kg or 0.1-3 mg/kg, for example about 0.5-2 mg/kg.

In accordance with the foregoing the present disclosure provides in a yet further aspect:

A method as defined above comprising co-administration, e.g., concomitantly or in sequence, of a therapeutically effective amount of an inhibitory BTN3A antibody of the disclosure (e.g. mAb1), and at least one second drug substance, said second drug substance being an anti-viral, anti-inflammatory, or anti-microbial agent, e.g. as indicated above.

In one embodiment, the antibodies of the disclosure may also be used to detect levels of BTN3A expressing cells. This can be achieved, for example, by incubating a sample (such as an in vitro sample) and a control sample with the anti-BTN3A antibody under conditions that allow for the formation of a complex between the antibody and BTN3A (as expressed at the surface of the cells, for example in a blood sample). Any complexes formed between the antibody and BTN3A are detected and compared in the sample and the control. For example, standard detection methods, well known in the art, such as ELISA and flow cytometric assays, can be performed using the compositions of the disclosure.

Accordingly, in one aspect, the disclosure further provides methods for detecting the presence of BTN3A, or BTN3A expressing cells (e.g., human BTN3A antigen) in a sample, or measuring the amount of BTN3A, comprising incubating the sample, and a control sample, with an antibody of the disclosure, which specifically binds to BTN3A, under conditions that allow for formation of a complex between the antibody and BTN3A. The formation of a complex is then detected, wherein a difference in complex formation between the sample compared to the control sample is indicative of the presence of BTN3A in the sample.

Also, within the scope of the present disclosure are kits consisting of the compositions (e.g., mAb1) disclosed herein and instructions for use. The kit can further contain a least one additional reagent, or one or more additional antibodies or proteins. Kits typically include a label indicating the intended use of the contents of the kit. The term label includes any writing, or recorded material supplied on or with the kit, or which otherwise accompanies the kit. The kit may further comprise tools for diagnosing whether a patient belongs to a group that will respond to an inhibitory BTN3A antibody treatment, as defined above.

The disclosure will be further illustrated by the following figures and examples. However, these examples and figures should not be interpreted in any way as limiting the scope of the present invention.

### EXAMPLES

Functional Assays for Selecting Candidate BTN3A Antibodies Having γδ Inhibitory Properties for their Use in Treating Gastro-Intestinal Inflammatory Disorders.

### SPR Biacore Assay

Multi-cycle kinetic analysis can be performed on a candidate BTN3A, using a Biacore T200 (serial no. 1909913) instrument running Biacore T200 Evaluation Software V2.0.1 (Uppsala, Sweden).

The purified candidates are diluted to a concentration of 2 μg/ml in 2% BSA/PBS. At the start of each cycle, each antibody is captured on the Protein A at a density (RL) of ˜146.5 RU (theoretical value to obtain an RMax of ˜50 RU). Following capture, the surface is allowed to stabilize before injection of the BTN3A1 antigen (Sino Biological cat. no. 15973-H08H). BTN3A1 is titrated in 0.1% BSA/HBS-P+(running buffer) in a two-fold dilution range from 25 to 0.78 nM. The association phase is monitored for 400 seconds and the dissociation phase for 35 minutes (2100 seconds). Kinetic data are obtained using a flow rate of 50 μl/min to minimize any potential mass transfer effects. Regeneration of the Protein A surface is conducted using two injections of 10 mM glycine-HCL pH 1.5 at the end of each cycle. Two blanks (no BTN3A1) and a repeat of a single concentration of the analyte can be performed for each tested antibody to check the stability of the surface and analyte over the kinetic cycles. The signal from the reference channel Fc1 is subtracted from that of Fc2, Fc3 and Fc4 to correct for differences in non-specific binding to a reference surface. Additionally, blank runs are subtracted for each Fc to correct any antigen-independent signal variation, such as drift. Sensorgrams are fitted using a one-to-one binding mathematical model with a global RMax parameter and no bulk signal (Constant RI=0 RU).

### PBMC Binding Assay

Human PBMCs were isolated from healthy community donor buffy coats (from blood drawn within 24 hours) using Lymphoprep (Axis-shield, Dundee, UK) density centrifugation. Cyno whole blood was obtained from Envigo (Huntingdon, UK). The day of the experiment, frozen PBMCs were revived and counted. Cells were stained for viable and dead cells by incubating for 30 min in the dark using the LIVE/DEAD® Far Red Dead Cell staining kit (ThermoFisher, Paisley, UK) following the manufacturer's instructions. During this staining, a 1 in 3 titration curve of test antibody was prepared in Flow buffer (0.5% BSA, 2 mM EDTA, 1×DPBS, pH 7.4) in a 96-well dilution plate. Following viability staining, the cells were harvested, washed twice and resuspended at 1×10⁶ cells/mL in Flow buffer. 100 μL cells at 1×10⁶ cells/mL was transferred to each well of a fresh U-shaped bottom 96-well plate. The plate was centrifuged, supernatant discarded and the cells were resuspended in 50 μL of the diluted test antibody titration series previously prepared. After incubation for 30 minutes at 4° C. in the dark, the plate was centrifuged, washed twice and resuspended in 50 μL goat anti-human antibody, PE labelled (Sigma, Poole, UK) diluted 1/100 in Flow buffer. After incubation for 15 minutes at 4° C. in the dark, the plate was centrifuged, washed and the cells were resuspended in 200 μL Flow buffer. Cells were then analysed on an Attune NxT focusing cytometer (ThermoFisher Scientific, Loughborough, UK), collecting 10,000 events per sample using two laser channels: RL1 for live/dead cells and BL2 for PE. Data was analysed using the instrument statistic tool or FlowJo software (Version 10, FlowJo, LLC, Ashland, USA) gating on the live population of lymphocyte cells. The X-median values from BL2 channel (PE signal) were then calculated and plotted against concentration.

### CD107 Degranulation Assay

The assay consists of measuring inhibitory effect of BTN3A candidate antibody on γδ T cell degranulation ability against Daudi Burkitt's lymphoma cell line (Harly C et al., Blood, 2012, Vol. 120 Issue 11 Pages 2269-79). γδ T cells are expanded from PBMCs of healthy donors by culturing them with zoledronic acid (1 μM) and IL2 (200 IU/ml) for 11-13 days. IL2 is added at days 5, 8 and every 2 days thereafter. The percentage of γδ T cells is determined at the initiation of culture and assessed for the time of culture by flow cytometry until it reaches at least 80%. Frozen γδ-T cells are then used in degranulation assays against Daudi cell line (E:T ratio of 1:1), whereby the cells are co-cultured for 4 hours at 37° C. in presence of increasing concentrations of the candidate antibody or control version. Activation by PMA (20 ng/ml) plus lonomycin (1 μg/ml) served as positive control for γδ T cell degranulation, and medium alone as negative control. At the end of the 4 hour incubation, cells are analyzed by flow cytometry to evaluate the percentage of γδ T cells positive for CD107a (LAMP-1, lysosomal-associated membrane protein-1)+CD107b (LAMP-2). CD107 is mobilized to the cell surface following activation-induced granule exocytosis, thus measurement of surface CD107 is a sensitive marker for identifying recently degranulated cytolytic T cells.

Ex Vivo Assay for Vδ2+ T Cell Activation from PBMC

PBMC from blood of Crohn's Disease or Ulcerative Colitis patients are isolated, stained with 1 μM CTV dye and cultured for 4 days in the presence of IL-2 (20 IU/ml)/IL-15 (20 ng/ml), 1 nM HDMAPP or HMBPP (phosphoantigen) and 1 μg/ml control isotype or the candidate BTN3A antibody.

Expression of the following markers of T cell activation: CD25, HLA-DR, β7 integrin, CD49b can be assessed for Vδ2+ and Vδ2− cells by flow cytometry.

Proliferation can be assessed by measuring CTV dilution and degranulation ability can be by measuring CD107a expression of Vδ2+ and Vδ2− also by flow cytometry.

Ex Vivo Assay for Vδ2+ T Cell Activation from Gut Biopsy

Gut biopsies from Crohn's Disease of Ulcerative Colitis patients are put in culture for “walk out” overnight culture. Biopsies are removed the day after, and the walkout cells are then stained with 1 μM of CTV dye. CTV-stained cells are then incubated 7 days in the presence of IL-2 (20 IU/ml)/IL-15 (20 ng/ml), 10 nM HDMAPP or HMBPP (phosphoantigen) and 1 μg/ml control isotype or the candidate BTN3A antibody. Expression of the following markers of T cell activation: CD25, HLA-DR can be assessed for Vδ2+ by flow cytometry. Proliferation can be assessed by measuring CTV dilution and degranulation ability can be measured by CD107a expression of Vδ2+ also by flow cytometry.

### Example 1: Generation of Inhibitory BTN3A1 Suitable for Use as a Drug in Human Subject

### Design of Composite Human Antibody™ Variable Region Sequence

Structural models of the murine 103.2 antibody V region (WO201280351) was produced using Swiss PDB and analyzed in order to identify important “constraining” amino acids in the V regions that were likely to be essential for the binding properties of the antibody.

Most residues contained within the CDRs (using both Kabat and Chothia definitions) together with several framework residues were considered to be important. The VH and Vκ sequences of murine 103.2 mAb contain typical framework residues and the CDR 1, 2 and 3 motifs are comparable to many murine antibodies.

Based upon the structural analysis, a large preliminary set of sequence segments that could be used to create 103.2 humanized variants were selected and analyzed using iTope™ technology for in silico analysis of peptide binding to human MHC class II alleles (Perry et al., 2008, Drugs R D 9 (6): 385-396), and using the TCED™ of known antibody sequence-related T cell epitopes (Bryson et al., 2010, Biodrugs 24 (1):1-8). Sequence segments that were identified as significant non-human germline binders to human MHC class II or that scored significant hits against the TCED™ were discarded. This resulted in a reduced set of segments, and combinations of these were again analyzed, as above, to ensure that the junctions between segments did not contain potential T cell epitopes. Selected sequence segments were assembled into complete V region sequences predicted to be devoid of significant T cell epitopes.

Five heavy chain (VH1 to VH5) and four light chain (Vκ1 to Vκ4) sequences were then chosen for gene synthesis and expression in mammalian cells.

### Construction of Humanized Variants Plasmids

The 20 humanized variants combining each of the 5 VH regions with each of the 4 Vk regions were synthesized with flanking restriction enzyme sites for cloning into an expression vector system for human IgG4 (S241P, L248E) heavy and kappa light chains. All constructs were confirmed by sequencing.

To assess the binding of all Composite Human Antibody™ variants and to select antibodies with suitable affinity to BTN3A as compared to the original murine antibody, single cycle kinetic analysis was performed on supernatants from transfected cell culture using a Biacore T200 (serial no. 1909913) running Biacore T200 Evaluation Software V2.0.1 (Uppsala, Sweden).

The results are provided hereafter in Table 1:

TABLE 1
Summary binding data for humanized antibody variants as determined
by single cycle kinetic analysis. Relative K_{D} values of humanised variants
were calculated by dividing the K_{D} of the humanised variant by that
of the chimeric antibody assayed in the same experiment

| | | Relative K_{D} to |
|---|---|---|
| Variant | K_{D} (M) | 103.2 chimeric |
| VH0/Vκ0 | 4.03 × 10⁻¹¹ | 1.0 |
| VH0/Vκ1 | 1.93 × 10⁻¹¹ | 0.5 |
| VH1/Vκ0 | 6.86 × 10⁻¹¹ | 1.7 |
| VH1/Vκ1 | 4.69 × 10⁻¹¹ | 1.2 |
| VH1/Vκ2 | 4.05 × 10⁻¹¹ | 1.0 |
| VH1/Vκ3 | 5.36 × 10⁻¹¹ | 1.3 |
| VH1/Vκ4 | 3.58 × 10⁻¹¹ | 0.9 |
| VH2/Vκ1 | 7.11 × 10⁻¹¹ | 1.8 |
| VH2/Vκ2 | 5.88 × 10⁻¹¹ | 1.5 |
| VH2/Vκ3 | 4.27 × 10⁻¹¹ | 1.1 |
| VH2/Vκ4 | 6.07 × 10⁻¹¹ | 1.5 |
| VH3/Vκ1 | 5.35 × 10⁻¹¹ | 1.3 |
| VH3/Vκ2 | 5.64 × 10⁻¹¹ | 1.4 |
| VH3/Vκ3 | 4.87 × 10⁻¹¹ | 1.2 |
| VH3/Vκ4 | 4.66 × 10⁻¹¹ | 1.2 |
| VH4/Vκ1 | 7.92 × 10⁻¹¹ | 2.0 |
| VH4/Vκ2 | 5.56 × 10⁻¹¹ | 1.4 |
| VH4/Vκ3 | 7.72 × 10⁻¹¹ | 1.9 |
| VH4/Vκ4 | 5.40 × 10⁻¹¹ | 1.3 |
| VH5/Vκ1 | 5.53 × 10⁻¹¹ | 1.4 |
| VH5/Vκ2 | 8.09 × 10⁻¹¹ | 2.0 |
| VH5/Vκ3 | 5.68 × 10⁻¹¹ | 1.4 |
| VH5/Vκ4 | 7.58 × 10⁻¹¹ | 1.9 |

Based on the affinities calculated by Biacore, as well as the iTope™ score and percentage of humanness of each humanized variant, six humanized variants with the closest affinities to the chimeric antibody and best iTope™ scores were selected for further analysis.

The selected humanized variants together with their chimeric version and the most conservatively humanized variant (VH1/Vκ1) were subjected to purification for further assay testing. Antibodies were purified from cell culture supernatants on Protein A sepharose columns followed by Size Exclusion Chromatography (SEC) (GE Healthcare, Little Chalfont, UK) using 10 mM sodium acetate, 100 mM NaCl, pH 5.5 as mobile phase and final formulation buffer. Samples were quantified by OD₂₈₀ₙₘ using an extinction coefficient (Ec(0.1%)) based on the predicted amino acid sequence.

Antibodies were analyzed using SDS-PAGE by loading 2 μg of each antibody on the gel and bands corresponding to the profile of a typical antibody were observed.

### Thermostability Analysis

To assess the thermostability of the six selected Composite Human Antibody™ variants, melting temperatures (the temperature at which 50% of a protein domain is unfolded) were determined using a fluorescence-based thermal shift assay.

All six purified humanized antibodies, together with the chimeric (VH0/Vκ0) antibody and the humanized variant (VH1/Vκ1), were diluted to a final concentration of 0.1 mg/ml in formulation buffer (10 mM sodium acetate, 100 mM NaCl, pH 5.5) containing SYPRO® Orange (ThermoFisher, Loughborough, UK) at 1 in 1000 dilution and subjected to a temperature gradient from 25° C. to 99° C. on a StepOnePlus real-time PCR system (ThermoFisher, Loughborough, UK) over a period of 56 minutes. 10 mM sodium acetate, 100 mM NaCl, pH 5.5 was used as a negative control. The melting curves were analyzed using protein thermostability software (version 1.2).

All antibody variants showed two distinct unfolding events with the higher Tₘ increasing as the degree of humanness increased as presented in Table 2.

TABLE 2
Melting temperature for the chimeric antibody,
humanized variant VH1/Vκ1 and the six 103.2
Composite Human Antibody ™ variants.

| | Single/Double | Tₘ1 mean | Tₘ2 mean |
|---|---|---|---|
| Variant | peaks | (° C.) | (° C.) |
| VH0/Vκ0 (chimeric) | Double | 61.4 | 71.3 |
| VH1/Vκ1 | Double | 61.4 | 72.5 |
| VH4/Vκ2 | Double | 61.4 | 76.5 |
| VH4/Vκ3 | Double | 61.4 | 76.9 |
| VH4/Vκ4 | Double | 61.6 | 77.6 |
| VH5/Vκ2 | Double | 61.4 | 78.5 |
| VH5/Vκ3 | Double | 61.5 | 78.7 |
| VH5/Vκ4 | Double | 61.3 | 79.5 |

### Multi-Cycle Kinetic Analysis

Multi-cycle kinetic analysis was performed on the six selected humanized 103.2 variants (VH4/Vκ2, VH4/Vκ3, VH4/Vκ4, VH5/Vκ2, VH5Vκ3, VH5/Vκ4) together with the chimeric antibody and the humanized variant, VH1/Vκ1, using a Biacore T200 (serial no. 1909913) instrument running Biacore T200 Evaluation Software V2.0.1 (Uppsala, Sweden).

Purified antibodies were diluted to a concentration of 2 μg/ml in 2% BSA/PBS. At the start of each cycle, each antibody was captured on the Protein A at a density (RL) of ˜146.5 RU (theoretical value to obtain an RMax of ˜50 RU). Following capture, the surface was allowed to stabilize before injection of the BTN3A1 antigen (Sino Biological cat. no. 15973-H08H). BTN3A1 was titrated in 0.1% BSA/HBS-P+(running buffer) in a two-fold dilution range from 25 to 0.78 nM. The association phase was monitored for 400 seconds and the dissociation phase for 35 minutes (2100 seconds). Kinetic data was obtained using a flow rate of 50 μl/min to minimize any potential mass transfer effects. Regeneration of the Protein A surface was conducted using two injections of 10 mM glycine-HCL pH 1.5 at the end of each cycle. Two blanks (no BTN3A1) and a repeat of a single concentration of the analyte were performed for each tested antibody to check the stability of the surface and analyte over the kinetic cycles. The signal from the reference channel Fc1 was subtracted from that of Fc2, Fc3 and Fc4 to correct for differences in non-specific binding to a reference surface. Additionally, blank runs were subtracted for each Fc to correct any antigen-independent signal variation, such as drift. Sensorgrams were fitted using a one-to-one binding mathematical model with a global RMax parameter and no bulk signal (Constant RI=0 RU).

The relative K_{D} compared to 103.2 chimeric (VH0/Vκ0) was calculated by dividing the K_{D} of the 103.2 Composite Human Antibody™ variants by that of the chimeric on the same chip. All selected Composite Human Antibody™ variants demonstrated affinity within two-fold of the chimeric antibody (Table 3).

TABLE 3
Multi cycle kinetic data for the binding of the six selected BT3.1-103.2
Composite Human Antibody ™ variants as well as the chimeric (VH0/Vκ0)
antibody and the humanised variant, VH1/Vκ1, to CD277 as determined using
the Biacore T200. The relative KD compared to chimeric (VH0/Vκ0) was
calculated by dividing the KD of the BT3.1-103.2 Composite Human Antibody ™ variants
by that of the BT3.1-103.2 chimeric assayed on the same chip.

| | kₐ | k_{d} | K_{D} | Relative K_{D} | R_{Max} | Chi² |
|---|---|---|---|---|---|---|
| Variant | (1/Ms) | (1/s) | (M) | to chimeric | (RU) | (RU²) |
| VH0/Vκ0 (chimeric) | 8.45 × 10⁵ | 8.98 × 10⁻⁵ | 1.06 × 10⁻¹⁰ | 1.0 | 65.6 | 0.466 |
| VH1/Vκ1 | 7.74 × 10⁵ | 8.80 × 10⁻⁵ | 1.14 × 10⁻¹⁰ | 1.1 | 59.0 | 0.279 |
| VH4/Vκ2 | 6.53 × 10⁵ | 9.71 × 10⁻⁵ | 1.49 × 10⁻¹⁰ | 1.4 | 72.9 | 0.367 |
| VH4/Vκ3 | 7.36 × 10⁵ | 9.51 × 10⁻⁵ | 1.29 × 10⁻¹⁰ | 1.2 | 56.1 | 0.232 |
| VH4/Vκ4 | 7.70 × 10⁵ | 8.33 × 10⁻⁵ | 1.08 × 10⁻¹⁰ | 1.0 | 57.8 | 0.245 |
| VH5/Vκ2 | 7.27 × 10⁵ | 9.42 × 10⁻⁵ | 1.30 × 10⁻¹⁰ | 1.2 | 65.4 | 0.293 |
| VH5/Vκ3 | 7.70 × 10⁵ | 1.03 × 10⁻⁴ | 1.33 × 10⁻¹⁰ | 1.3 | 53.3 | 0.260 |
| VH5/Vκ4 | 7.72 × 10⁵ | 9.55 × 10⁻⁵ | 1.24 × 10⁻¹⁰ | 1.2 | 55.2 | 0.250 |

### In Vitro Functional Efficacy: γδ-T Cell Degranulation Assay

The assay consists of measuring inhibitory effect of 103.2 humanized variants and their chimeric version on γδ T cell degranulation ability against Daudi Burkitt's lymphoma cell line (Harly et al., 2012, see supra). γδ T cells were expanded from PBMCs of healthy donors by culturing them with zoledronic acid (1 μM) and IL2 (200 IU/ml) for 11-13 days. IL2 was added at days 5, 8 and every 2 days thereafter. The percentage of γδ T cells was determined at the initiation of culture and assessed for the time of culture by flow cytometry until it reached at least 80%. Frozen γδ T cells were then used in degranulation assays against Daudi cell line (E:T ratio of 1:1), whereby the cells were co-cultured for 4 hours at 37° C. in presence of increasing concentrations of the 103.2 humanized variants and their chimeric and mouse versions. Activation by PMA (20 ng/ml) plus lonomycin (1 μg/ml) served as positive control for γδ T cell degranulation, and medium alone as negative control. At the end of the 4 hours incubation, cells were analyzed by flow cytometry to evaluate the percentage of γδ T cells positive for CD107a (LAMP-1, lysosomal-associated membrane protein-1)+CD107b (LAMP-2). CD107 is mobilized to the cell surface following activation-induced granule exocytosis, thus measurement of surface CD107 is a sensitive marker for identifying recently degranulated cytolytic T cells.

All tested humanized variants kept their inhibitory effect in the degranulation assay as compared to the chimeric and mouse antibodies (see Table 4).

Surprisingly VH4Vκ4 which included an amino acid mutation in CDR2 as compared to the original murine CDR2 of murine mAb 103.2 was still very potent in this assay.

TABLE 4
IC50 of various BTN3A inhibitory antibodies derived from mAb 103.2

| | m103.2 | H0K0 | H1K1 | H4K2 | H4K3 | H4K4 | H5K2 | H5K3 | H5K4 |
|---|---|---|---|---|---|---|---|---|---|
| IC50 | 0.0056 | 6.29 × 10⁻¹⁰ | 1.77 × 10⁻⁷ | 0.0036 | 1.087 × 10⁻⁷ | 0.0121 | 0.00483 | 0.0015 | 0.0062 |
| (μg/ml) | | | | | | | | | |
| span | 27.60 | Unstable | Unstable | 21.65 | 2447 | 12.11 | 24.58 | 35.43 | 22.76 |

### Selection of Humanized Candidates

Altogether, it was surprisingly noticed that VH4Vκ4 humanized variant presented the highest Biacore affinity as well as the highest binding affinity to human PBMC and was the most potent for inhibitory properties in the degranulation assay at a concentration of 10 μg/ml as compared to the other humanized candidates.

In addition, the thermostability of VH4Vκ4 was even improved compared to the chimeric antibody with murine VH and VL regions.

Accordingly, the humanized candidate VH4Vκ4 was selected for further evaluation in vivo and in vitro for its suitability for treating IBD.

### Cross-Reactivity on Human and Cynomolgus PBMCs

Human PBMCs were isolated from healthy community donor buffy coats (from blood drawn within 24 hours) obtained under consent from commercial vendors. Cyno whole blood was obtained from Envigo (Huntingdon, UK). PBMCs were isolated using Lymphoprep (Axis-shield, Dundee, UK) density centrifugation. PBMCs were then frozen and stored in the vapor phase of nitrogen until required.

Frozen PBMCs were revived, added to pre-warmed AIM-V (ThermoFisher Scientific, Loughborough, UK) media and counted. Cells were harvested, washed by centrifuging at 500×g for 10 minutes and then resuspended in 1×DPBS, pH 7.4. This step was repeated once and the cells were resuspended in 1×DPBS, pH 7.4 at 1×10⁶ cells/mL. Cells were stained for viable and dead cells by incubating for half an hour in the dark using the LIVE/DEAD® Far Red Dead Cell staining kit (ThermoFisher, Paisley, UK) following the manufacturer's instructions. An aliquot of cells heated at 70° C. for ten minutes was included as the dead cell control. During this staining, a three-point, 1 in 3 titration curve of test antibody (starting from 0.063 μg/mL for the selected antibodies) was prepared in Flow buffer (0.5% BSA, 2 mM EDTA, 1×DPBS, pH 7.4) in a 96-well dilution plate. Following viability staining, the cells were harvested, washed twice as described previously and then resuspended at 1×10⁶ cells/mL in Flow buffer. 100 μL cells at 1×10⁶ cells/mL was transferred to each well of a fresh U-shaped bottom 96-well plate (columns 1-12). The plate was centrifuged, supernatant discarded and the cells were resuspended in 50 μL of the diluted test antibody titration series that was previously prepared. After incubation for 30 minutes at 4° C. in the dark, the plate was centrifuged, washed twice with 150 μL/well Flow buffer and the cells were resuspended in 50 μL goat anti-human antibody, PE labelled (Sigma, Poole, UK) diluted 1/100 in Flow buffer. After incubation for 15 minutes at 4° C. in the dark, the plate was centrifuged, washed once with 150 μL/well Flow buffer and the cells were resuspended in 200 μL Flow buffer. Cells were then analysed on an Attune NxT focusing cytometer (ThermoFisher Scientific, Loughborough, UK), collecting 10,000 events per sample using two laser channels: RL1 for live/dead cells and BL2 for PE. Data was analysed using the instrument statistic tool or FlowJo software (Version 10, FlowJo, LLC, Ashland, USA) gating on the live population of lymphocyte cells. The X-median values from BL2 channel (PE signal) were then calculated and plotted against concentration.

A full titration was then performed with an 11-point, 1 in 3 full titration curve of test antibody (starting from 5 μg/mL).

The Table 5 below show that binding was observed for both tested antibodies to both human and cynomolgus PBMCs. The pattern of binding was consistent between the two species as well as between the different donors.

TABLE 5
summary table of EC50 values (μg/mL) for the binding
of chimeric and corresponding lead humanized antibodies
to human or cynomolgus PBMCs and of theoretical maximum
binding based on the theoretical predicted curves.

| | Human | Cyno | Cyno | Cyno |
|---|---|---|---|---|
| | PBMCs | PBMCs | PBMCs | PBMCs |
| | Donor 1 | Donor 1 | Donor 2 | Donor 3 |
| EC50 | | | | |
| 103.2 VH0/V κ 0 | 0.019 | 0.014 | 0.012 | 0.013 |
| 103.2 VH4/V κ 4 | 0.017 | 0.013 | 0.011 | 0.012 |
| Theoretical Max | | | | |
| binding (MFI) | | | | |
| 103.2 VH0/V κ 0 | 19640 | 8950 | 5942 | 5146 |
| 103.2 VH4/V κ 4 | 19783 | 8560 | 5821 | 4968 |

### Epitope Mapping

To determine the epitope of the antibody/antigen complexes with high resolution, the protein complexes were incubated with deuterated cross-linkers and subjected to multi-enzymatic cleavage. After enrichment of the cross-linked peptides, the samples were analyzed by high resolution mass spectrometry (nLC-Q Exactive MS) and the data generated were analyzed using XQuest and Stavrox software.

A mixture of BTN3A1/mAb1 was prepared with the following concentrations:

| | BTN3A1 | | Antibody | | BTN3A1/Antibody | |
|---|---|---|---|---|---|---|
| mixture | volume | conc | volume | conc | volume | conc |
| BTN3A1/mAb1 | 10 μl | 4.48 μM | 10 μl | 2.12 μM | 20 μl | 2.24 μM/1.06 μM |

For reduction alkylation, 20 μL of the Antibody/Antigen mixture prepared were mixed with 2 μL of DSS d0/d12 (2 mg/mL; DMF) before 180 minutes incubation time at room temperature. After incubation, reaction was stopped by adding 1 μL of Ammonium Bicarbonate (20 mM final concentration) before 1 h incubation time at room temperature.

Then, the solution was dried using a speedvac before H₂O 8M urea suspension (20 μL). After mixing, 2 μl of DTT (500 mM) were added to the solution. The mixture was then incubated 1 hour at 37° C. After incubation, 2 μl of iodoacetamide (1M) were added before 1 hour incubation time at room temperature, in a dark room. After incubation, 80 μl of the proteolytic buffer were added. The trypsin buffer contains 50 mM Ambic pH 8.5, 5% acetonitrile. The chymotrypsin buffer contains Tris HCl 100 mM, CaCl₂ 10 mM pH 7.8. The ASP-N buffer contains Phosphate buffer 50 mM pH 7.8. The elastase buffer contains Tris HCl 50 mM pH 8.0 and the thermolysin buffer contains Tris HCl 50 mM, CaCL2 0.5 mM pH 9.0.

For trypsin proteolysis, 100 μl of the reduced/alkyled antibody/antigen mixtures were mixed with 0.9 μl of trypsin (Roche Diagnostic) with the ratio 1/100 (w/w). The proteolytic mixtures were incubated overnight at 37° C.

For chymotrypsin proteolysis, 100 μl of the reduced/alkyled antibody/antigen mixture were mixed with 0.45 μl of chymotrypsin (Roche Diagnostic) with the ratio 1/200 (w/w). The proteolytic mixtures were incubated overnight at 25° C.

For ASP-N proteolysis, 100 μl of the reduced/alkyled antibody/antigen mixtures were mixed with 0.45 μl of AspN (Roche Diagnostic) with the ratio 1/200 (w/w). The proteolytic mixtures were incubated overnight at 37° C.

For the elastase proteolysis, 100 μl of the reduced/alkyled antibody/antigen mixtures were mixed with 0.9 μl of elastase (Roche Diagnostic) with the ratio 1/100 (w/w). The proteolytic mixtures were incubated overnight at 37° C.

For the thermolysin proteolysis, 100 μl of the reduced/alkyled antibody/antigen mixtures were mixed with 1.8 μl of thermolysin (Roche Diagnostic) with a ratio 1/50 (w/w). The proteolytic mixtures were incubated overnight at 70° C. After digestion formic acid 1% final was added to the solution

After Trypsin, Chymotrypsin, ASP-N, Elastase and Thermolysin proteolysis of the antibody/antigen protein complex with deuterated d0d12, the nLC-orbitrap MS/MS analysis detected five cross-linked peptides between BTN3A1 (protein 2) and mAb1 (protein 1). The sequences and positions of cross-links are presented in Table 6 below:

TABLE 6
Cross-linked peptides detected between BTN3A1 and mAb1.

| | | Sequence | Sequence | | | | Identified |
|---|---|---|---|---|---|---|---|
| Sequence | Enzyme | Protein 1 | Protein 2 | XLType | nAA1 | nAA2 | on StavroX |
| VINPRSGDSHYNEKFKDR- | Thermolysin | 50-67 | 48-57 | inter- | 58 | 53 | YES |
| ADLPCHLFPT-a9-b6 | | | | protein xl | | | |
| AFTSYL-ADLPCHLFPTMS- | Thermolysin | 28-33 | 48-59 | inter- | 30 | 53 | YES |
| a3-b6 | | | | protein xl | | | |
| INPRSGDSHYNEKFKDRVT- | Thermolysin | 51-69 | 58-64 | inter- | 60 | 62 | YES |
| MSAETME-a10-b5 | | | | protein xl | | | |
| VINPRSGDSHYNEKFKDR- | Thermolysin | 50-67 | 63-67 | inter- | 59 | 66 | YES |
| MELKW-a10-b4 | | | | protein xl | | | |

Using chemical cross-linking, High-Mass MALDI mass spectrometry and nLC-Orbitrap mass spectrometry we were able to characterize the epitope of mAb1 on BTN3A1.

As shown in FIG. 1, the epitope includes the following amino acids on the antigen BTN3A1: 53, 62, 66, as comprised in SEQ ID NO:20.

### Off Target

Cell microarray technology (Retrogenix) was used to screen for specific off-target binding of the Fc-silenced humanized 103.2 IgG1 (referred to as mAb1, which targets human BTN3A proteins.

Investigation of the levels of binding of the test antibody to untransfected HEK293 cells, and to cells over-expressing BTN3A1, before or after cell fixation, showed 2 μg/ml on fixed cells to be a suitable screening condition. Under this condition, the test antibody was screened for binding against human HEK293 cells, individually expressing 5528 human proteins, comprising of cell surface membrane proteins and cell surface-tethered secreted proteins. This revealed eleven primary hits. Each primary hit was re-expressed, along with two control receptors (CD20 and EGFR) and re-tested with 2 μg/ml test antibody, 2 μg/ml of an isotype control antibody, and other positive and negative control treatments. After removing one very weak intensity, non-reproducible hit and five non-specific hits, there remained five specific interactions for the test antibody. These were two isoforms of BTN3A1, two isoforms of BTN3A2 and one isoform of BTN3A3, its primary targets.

No off-target interactions for mAb1 were identified, indicating high specificity of mAb1 for its primary targets (Data not shown).

### Example 2: In Vitro Pharmacology

Assessment of Helper Functions of Peripheral Vγ9Vδ2 T Cells Upon Microbial Activation and Ability of mAb1 to Modulate Vγ9Vδ2 T Cells “Helper” Functions

Vγ9Vδ2 T cells are known to express several markers upon phosphoantigen stimulation, particularly in IBD (Mann E R et al, Clin Exp Immunol, 2012 November; 170(2):122-30; Tyler C J et al, J Immunol, 2017 May 1; 198(9):3417-3425; McCarthy N E et al, J Clin Invest, 2015 Aug. 3; 125(8):3215-25). To see if humanized 103.2 Fc-silenced IgG1 (hereafter referred as mAb1) could impact the phenotype of Vγ9Vδ2 T cells but also of other immune populations, PBMC from 6 healthy donors were stimulated with phosphoantigens (Hmbpp) at 0.5 μM+/−50 IU/ml IL-2 in presence of increasing concentrations of humanized 103.2 or corresponding control isotype for 2 days. Expression of costimulation markers (CD40, CD80 and CD86), mature APCs marker (CD83), antigen presentation molecules (HLA-DR), adhesion molecules (CD11a, CD11b, CD11c and CD54), gut and lymph node homing molecules (α4 and β7 integrins, CCR9 and CCR7) and B cell costimulatory molecules (OX40, ICOS and CD70) were assessed by flow cytometry on Vγ9Vδ2 T cells and other immune cells (B cells, monocytes and Vδ2− T cells) and IC50 were calculated.

mAb1 is inhibiting in a dose-dependent manner all markers evaluated (similar inhibition observed on the frequency of positive cells and median fluorescence intensity) indicating that mAb1 has the ability to inhibit APC function, gut homing potential and adhesion potential of peripheral Vγ9Vδ2 T cells from healthy donors stimulated with phosphoantigens.

Results are comparable in the absence or presence of IL-2 in the culture medium. Table 7 shows the different IC50 calculated through sigmoidal 4PL equation on the MFI of each marker.

TABLE 7
IC50 values

IC50 (μg/ml)

| function | marker | HmBPP | HmBPP + IL-2 |
|---|---|---|---|
| Antigen | HLA-DR | 0.006 | 0.003 |
| presentation and | CD40 | 0.004 | 0.005 |
| costimulation | CD80 | 0.002 | 0.004 |
| | CD83 | 0.002 | 0.004 |
| | CD86 | 0.005 | 0.003 |
| Gut and lymph | α4 integrin | 0.002 | 0.003 |
| node homing | β7 integrin | 0.003 | 0.005 |
| | CCR7 | 0.002 | 0.003 |
| | CCR9 | 0.006 | 0.005 |
| Adhesion | CD54 | 0.002 | 0.003 |
| molecules | CD11a | 0.006 | 0.005 |
| | CD11b | 0.02 | 0.01 |
| | CD11c | 0.006 | 0.005 |
| B cell | OX40 | 0.001 | 0.005 |
| costimulation | CD70 | 0.005 | 0.005 |
| | ICOS | 0.002 | 0.002 |

The impact of mAb1 was specific of Vδ2+ T cells and was not observed on other immune cell populations: Vδ2− T cells, B cells, monocytes. This provides good prospects for a safe use of mAb1 as an anti-inflammatory treatment, in particular in gastro-intestinal inflammatory disorders where activated δ2 T cells are involved.

Kinetic of the In Vitro Effect of mAb1 on Phosphoantigen Mediated-Vγ9Vδ2 T Cell Activation

PBMC from 3 healthy donors were stimulated with phosphoantigens (Hmbpp) at 0.5 μM+50 IU/ml IL-2 in presence of 10 μg/ml mAb1 or corresponding control isotype for the indicated timepoints. Same markers were evaluated by flow cytometry.

The results show that mAb1 is able to inhibit the expression of all markers evaluated from 24 hours after the initiation of the in vitro treatment until the end of the culture at day 6, indicating a long-lasting in vitro effect of the antibody.

Assessment of the Potential Impact of mAb1 on Different Peripheral Immune Compartments with or without Stimulation

PBMC from 3 healthy donors or whole blood depleted from red blood cells from 8 healthy donors were cultured with 10 μg/ml of Hu103.2 or corresponding control isotype with or without different stimuli for 2 days and 5 days. The stimuli used were phosphoantigens (HmBPP) known to activate Vγ9Vδ2 T cells; IL-15 known to activate Vγ9Vδ2 T cells, NK cells and CD8+αβ T cells; phytohemagglutinin known to activate lymphocytes, anti IgA/G/M known to activate B cells, TLR agonists (lipopolysaccharide known to activate monocytes, macrophages, dendritic cells and B cells, R848 known to activate myeloid cells, plasmacytoid dendritic cells and B cells, CpGB known to activate B cells and monocytes). Expression of HLA-DR (antigen presentation molecule), costimulation molecules (CD40 &CD86), activation markers (CD69 and CD25), CCR9 (gut homing receptor), CD11c (adhesion molecule) and proliferation ability of different peripheral immune cell compartments (Vγ9Vδ2 T cells, B cells, monocytes, NK cells, CD8+ and CD8− αβT cells) was assessed by flow cytometry after 2 days of culture for marker expression and 5 days for proliferation ability. Proliferation was evaluated using CTV-stained PBMC.

The results show that mAb1 inhibits the expression of all markers evaluated and the proliferation ability of Vγ9Vδ2 T cells only in response to phosphoantigen. Interestingly, the observed inhibition was specific to Vγ9Vδ2 T cells, as mAb1 is not affecting activation markers or proliferation ability of any of the other immune cell compartments evaluated in response to any of the stimuli tested.

These results indicate that mAb1 is able to specifically inhibit phosphoantigen-driven activation of Vγ9Vδ2 T cells. These results were confirmed using whole blood depleted from red blood cells from 8 healthy donors.

### Example 3: In Vitro and In Vivo Efficacy

mAb1 Inhibits Vγ9Vδ2 T Cell Activated Phenotype, Proliferation and Degranulation from IBD patients induced by phosphoantigens

PBMC from blood of Crohn's Disease or Ulcerative Colitis patients were isolated, stained with 1 μM CTV dye and cultured for 4 days in the presence of IL-2/IL-5, 1 nM HDMAPP (phosphoantigen) and 1 μg/ml control isotype or humanized 103.2 mAb. Expression of several markers (CD25, HLA-DR, β7 integrin, CD49b), proliferation and degranulation ability (as measured by CTV dilution and CD107a expression respectively) of Vδ2+ and Vδ2− were assessed by flow cytometry. The frequency of Vδ2+ T cells was comparable between non IBD and IBD individuals (data not shown). Peripheral Vδ2+ T cells from non IBD and IBD patients are responsive to phosphoantigens in a similar way, this being not affected when an isotype control is added in the culture.

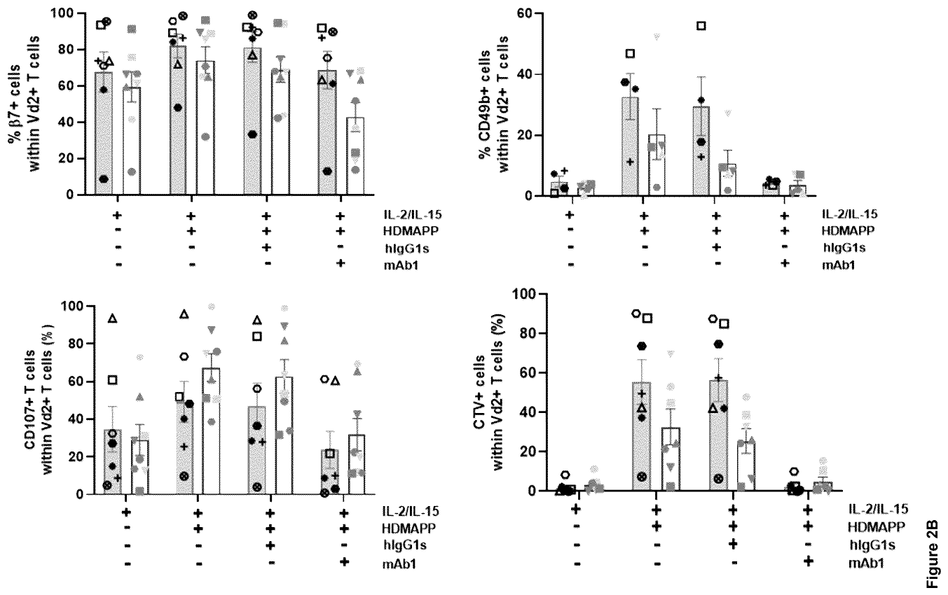

By contrast, the addition of mAb1 is inhibiting the phosphoantigen-mediated activation of Vδ2+ T cells from non IBD and IBD patients in a similar way (FIGS. 2A and B). This indicates that mAb1 can inhibit the ex vivo activation, gut homing potential, proliferation and degranulation ability of peripheral Vδ2+ T cells from IBD patients.

mAb1 has Thus the Potential to Decrease the Migration of Peripheral Vδ2+ T Cells to the Gut and Thus Decrease the Inflammatory Potential of Peripheral Vδ2+ T Cells.

These results were confirmed on gut biopsies from non IBD and IBD patients. Gut biopsies were put in culture for “walk out” overnight culture. Biopsies were removed the day after, and the walkout cells were stained with 1 μM of CTV dye. CTV-stained cells were then incubated 7 days in the presence of IL-2/IL-5, 10 nM HDMAPP (phosphoantigen) and 1 μg/ml control isotype or mAb1.

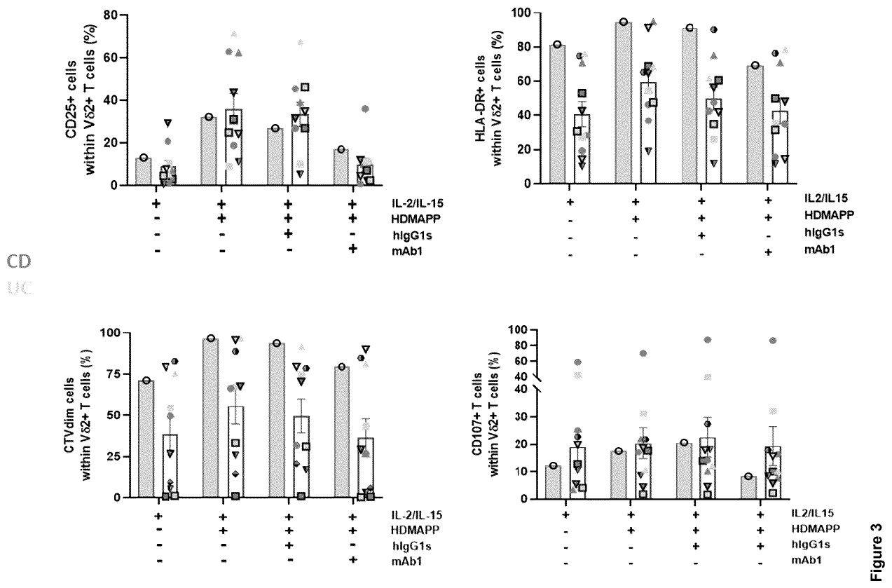

mAb1 was also able to inhibit the phosphoantigen-mediated activation of Vδ2+ T cells from gut biopsies of IBD patients which could lead to a decrease in the gut inflammation, dampening the disease (FIG. 3).

mAb1 Inhibits In Vivo the Clinical Signs Associated to DSS-Induced Colitis in Cynomolgus Monkeys

DSS-induced colitis model in non human primates is known to resemble features of gastro-intestinal disorders such as human ulcerative colitis, making this model a tool to study important immunological mechanisms and efficacy of novel treatments (Takahashi N et al, 2020, Heliyon, 6(1): e03178; McQueen P et al, 2019, Mucosal Immunol, 12(6): 1327).

BTN3A ortholog and Vγ9Vδ2 T cells are present in non-human primates. The therapeutic efficacy of mAb1 was thus tested in an acute DSS-induced colitis model in cynomolgus monkeys.

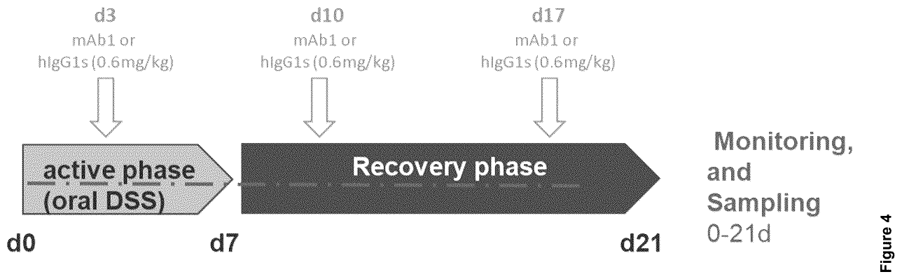

A total of 14 naïve male Cynomolgus monkeys (3-5 kg) were enrolled into two groups in this study. DSS was dissolved in saline to a concentration of 40 mg/mL, protected from light. From Day 0, each monkey received oral administration of 0.65 g DSS (dissolved in water) twice daily for 7 days. mAb1 or its corresponding isotype control were administered at days 3, 10 and 17 intravenously at a dose of 0.6 mg/kg. Body weight loss and disease activity index (DAI) were monitored daily throughout the study. The colitis clinical severity was assessed by Diarrhea (0, normal stool consistency; 1, Soft stools; 2 Mushy stools; and 3, Watery stools), Intestinal bleeding (0, no bleeding; 1, positive hemoccult; 2, visible bloody stool; 3, massive rectal bleeding) and body weight loss (0, ≤1%; 1, 1-5%; 2, 5-10%; 3, 10-15%; and 4, >15%). To calculate DAI, sum of the scores above was performed then divided by 3. Serum samples were collected at indicated timepoints and stored until cytokine analysis. Cytokine analysis was performed using the MSD technology and the V-Plex NHP Cytokine 24Plex kit. The study lasted until Day 21 (FIG. 4).

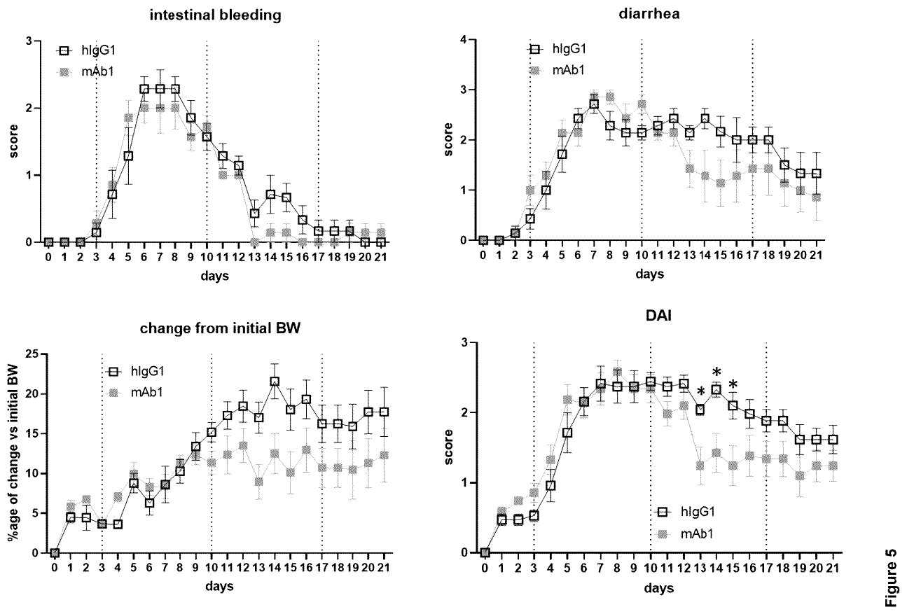

As shown in FIG. 5, clinical signs associated to DSS-induced colitis (intestinal bleeding, diarrhea, body weight loss, disease activity index) are reduced after injections of mAb1 compared to monkeys injected with the corresponding isotype control, indicating that mAb1 has potential therapeutic benefit in IBD treatment.

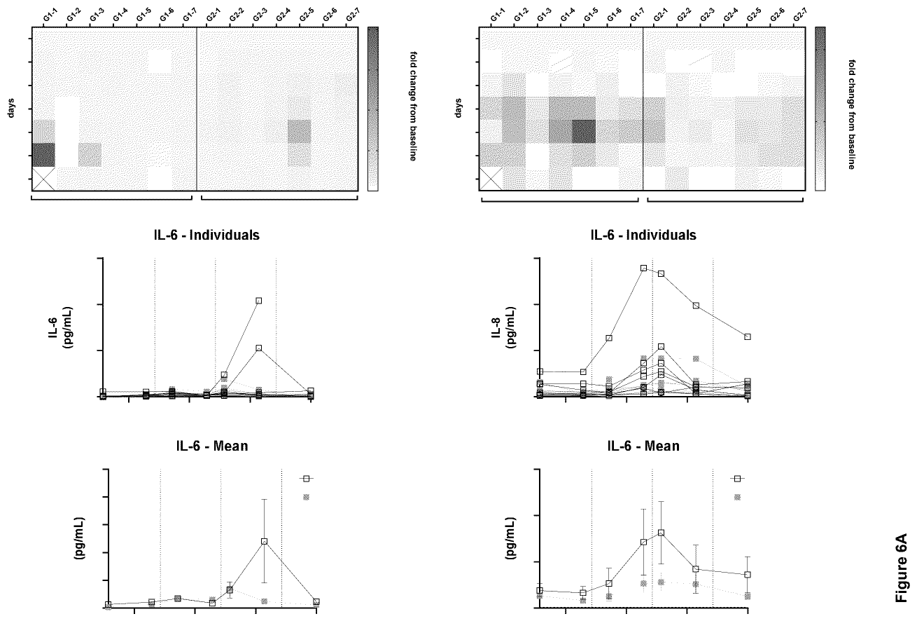

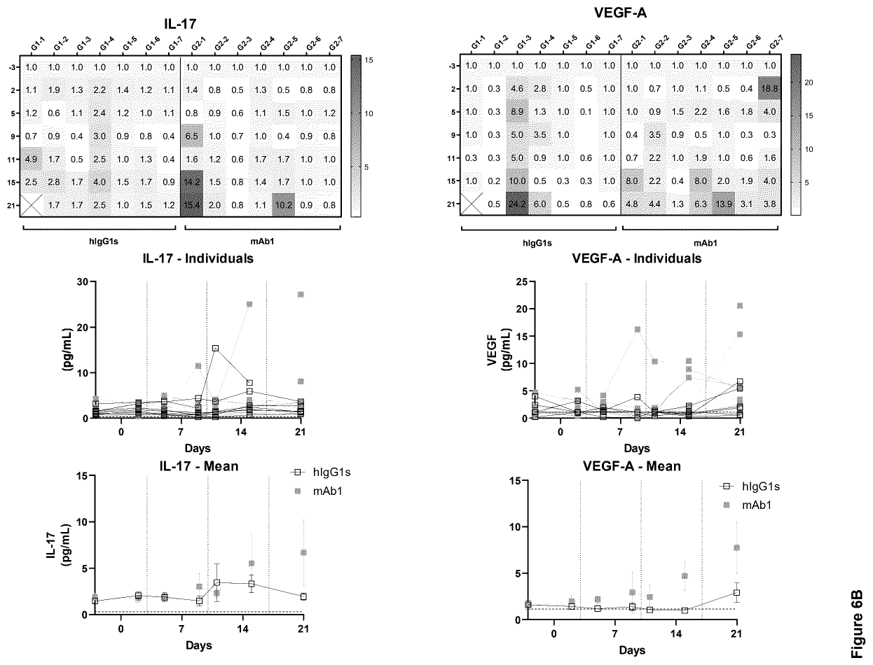

Moreover, as shown in FIG. 6, the circulating levels of pro-inflammatory cytokines IL-6 and IL-8 were lower in the animals treated with mAb1 compared to the animals treated with the isotype control. Concomitantly, the levels of circulating immunoprotective IL-17 and of VEGF were increased in the animals treated with mAb1 compared to the animals treated with the isotype control, confirming mAb1 may lead to a less inflammatory environment in IBD.

### SEQUENCE LISTING

Tables 8 and 9: Brief description of useful amino acid and nucleotide sequences for practicing the invention

TABLE 8

| SEQ ID | | |
|---|---|---|
| NO: | Type | Description of the sequence |
| 1 | aa | HCDR1 of mAb1 |
| 2 | aa | HCDR2 of mAb1 |
| 3 | aa | HCDR3 of mAb1 |
| 4 | aa | LCDR1 of mAb1 |
| 5 | aa | LCDR2 of mAb1 |
| 6 | aa | LCDR3 of mAb1 |
| 7 | aa | VH of mAb1 |
| 8 | aa | VL of mAb1 |
| 9 | nt | HCDR1 of mAb1 |
| 10 | nt | HCDR2 of mAb1 |
| 11 | nt | HCDR3 of mAb1 |
| 12 | nt | LCDR1 of mAb1 |
| 13 | nt | LCDR2 of mAb1 |
| 14 | nt | LCDR3 of mAb1 |
| 15 | nt | VH of mAb1 |
| 16 | nt | VL of mAb1 |
| 17 | aa | Human BTN3A1 |
| 18 | aa | Human BTN3A2 |
| 19 | aa | Human BTN3A3 |
| 20 | aa | Epitope of mAb1 on human BTN3A1 |
| 21 | aa | Full length heavy chain of mAb1 |
| 22 | aa | Full length light chain of mAb1 |
| 23 | aa | LCDR2 of murine 103.2 |
| 24 | aa | Cynomolgus BTN3A1 |
| 25 | aa | Epitope region on human BTN3A1 as shown in FIG. 1 |

TABLE 9

| SEQ | | |
|---|---|---|
| ID | | |
| NO: | Type | Description of the sequence |
| 1 | aa | SYLIH |
| 2 | aa | VINPRSGDSHYNEKFKD |
| 3 | aa | SDYGAY |
| 4 | aa | RASQSISNNLH |
| 5 | aa | YASQSAF |
| 6 | aa | QQSNSWPHT |
| 7 | aa | QVQMQQSGAEVKKPGASVKVSCKASGYAFTSYLIHWIKQRPGQG |
| | | LEWIGVINPRSGDSHYNEKFKDRVTMTADQSISTAYMELSRLRSDD |
| | | TAVYYCARSDYGAYWGQGTLVTVSS |
| 8 | aa | EIVLTQSPATLSVSPGERATLSCRASQSISNNLHWYQQKPGQAPR |
| | | LLIKYASQSAFGIPARFSGSGSGTEFTLTISSLQSEDFAVYFCQQSN |
| | | SWPHTFGQGTKLEIK |
| 9 | nt | AGTTACTTGATACAC |
| 10 | nt | GTGATTAATCCTAGAAGTGGTGATAGTCACTACAATGAGAAGTT |
| | | CAAGGAC |
| 11 | nt | TCAGATTACGGGGCTTAC |
| 12 | nt | AGGGCCAGCCAAAGTATTAGCAACAACCTACAC |
| 13 | nt | TATGCTTCCCAGTCCGCTTTT |
| 14 | nt | CAACAGAGTAACAGCTGGCCTCACACG |
| 15 | nt | CAGGTCCAGATGCAGCAGTCTGGAGCTGAGGTGAAGAAGCCTG |
| | | GGGCCTCAGTGAAGGTTTCCTGCAAGGCTTCTGGATACGCCTT |
| | | CACTAGTTACTTGATACACTGGATTAAACAGAGGCCTGGACAGG |
| | | GCCTTGAGTGGATTGGAGTGATTAATCCTAGAAGTGGTGATAGT |
| | | CACTACAATGAGAAGTTCAAGGACAGGGTCACAATGACTGCAG |
| | | ACCAGTCCATCAGCACTGCCTACATGGAGCTCAGCAGGCTGAG |
| | | ATCTGATGACACGGCGGTCTATTACTGTGCAAGATCAGATTACG |
| | | GGGCTTACTGGGGCCAAGGGACTCTGGTCACCGTCTCCTCA |
| 16 | nt | GAAATTGTGTTGACTCAGTCACCAGCCACCCTGTCTGTGTCTCC |
| | | AGGAGAAAGAGCCACCCTTTCCTGCAGGGCCAGCCAAAGTATT |
| | | AGCAACAACCTACACTGGTATCAGCAGAAACCTGGCCAGGCTC |
| | | CAAGGCTTCTCATCAAATATGCTTCCCAGTCCGCTTTTGGCATC |
| | | CCCGCCAGGTTCAGTGGCAGTGGATCAGGGACAGAATTCACTC |
| | | TCACCATCAGCAGTCTGCAGTCTGAAGATTTTGCAGTTTATTTCT |
| | | GTCAACAGAGTAACAGCTGGCCTCACACGTTCGGTCAGGGGAC |
| | | CAAGCTGGAGATCAAA |
| 17 | aa | MKMASFLAFLLLNFRVCLLLLQLLMPHSAQFSVLGPSGPILAMVGE |
| | | DADLPCHLFPTMSAETMELKWVSSSLRQVVNVYADGKEVEDRQS |
| | | APYRGRTSILRDGITAGKAALRIHNVTASDSGKYLCYFQDGDFYEK |
| | | ALVELKVAALGSDLHVDVKGYKDGGIHLECRSTGWYPQPQIQWSN |
| | | NKGENIPTVEAPVVADGVGLYAVAASVIMRGSSGEGVSCTIRSSLL |
| | | GLEKTASISIADPFFRSAQRWIAALAGTLPVLLLLLGGAGYFLWQQ |
| | | QEEKKTQFRKKKREQELREMAWSTMKQEQSTRVKLLEELRWRSI |
| | | QYASRGERHSAYNEWKKALFKPADVILDPKTANPILLVSEDQRSVQ |
| | | RAKEPQDLPDNPERFNWHYCVLGCESFISGRHYWEVEVGDRKE |
| | | WHIGVCSKNVQRKGWVKMTPENGFWTMGLTDGNKYRTLTEPRT |
| | | NLKLPKPPKKVGVFLDYETGDISFYNAVDGSHIHTFLDVSFSEALYP |
| | | VFRILTLEPTALTICPA |
| 18 | aa | MKMASSLAFLLLNFHVSLLLVQLLTPCSAQFSVLGPSGPILAMVGE |
| | | DADLPCHLFPTMSAETMELKWVSSSLRQVVNVYADGKEVEDRQS |
| | | APYRGRTSILRDGITAGKAALRIHNVTASDSGKYLCYFQDGDFYEK |
| | | ALVELKVAALGSNLHVEVKGYEDGGIHLECRSTGWYPQPQIQWSN |
| | | AKGENIPAVEAPVVADGVGLYEVAASVIMRGGSGEGVSCIIRNSLL |
| | | GLEKTASISIADPFFRSAQPWIAALAGTLPILLLLLAGASYFLWRQQK |
| | | EITALSSEIESEQEMKEMGYAATEREISLRESLQEELKRKKIQYLTR |
| | | GEESSSDTNKSA |
| 19 | aa | MKMASSLAFLLLNFHVSLFLVQLLTPCSAQFSVLGPSGPILAMVGE |
| | | DADLPCHLFPTMSAETMELRWVSSSLRQVVNVYADGKEVEDRQS |
| | | APYRGRTSILRDGITAGKAALRIHNVTASDSGKYLCYFQDGDFYEK |
| | | ALVELKVAALGSDLHIEVKGYEDGGIHLECRSTGWYPQPQIKWSDT |
| | | KGENIPAVEAPVVADGVGLYAVAASVIMRGSSGGGVSCIIRNSLLG |
| | | LEKTASISIADPFFRSAQPWIAALAGTLPISLLLLAGASYFLWRQQKE |
| | | KIALSRETEREREMKEMGYAATEQEISLREKLQEELKWRKIQYMAR |
| | | GEKSLAYHEWKMALFKPADVILDPDTANAILLVSEDQRSVQRAEEP |
| | | RDLPDNPERFEWRYCVLGCENFTSGRHYWEVEVGDRKEWHIGV |
| | | CSKNVERKKGWVKMTPENGYWTMGLTDGNKYRALTEPRTNLKLP |
| | | EPPRKVGIFLDYETGEISFYNATDGSHIYTFPHASFSEPLYPVFRILT |
| | | LEPTALTICPIPKEVESSPDPDLVPDHSLETPLTPGLANESGEPQAE |
| | | VTSLLLPAHPGAEVSPSATTNQNHKLQARTEALY |
| 20 | aa | HLFPTMSAETMELK |
| 21 | aa | QVQMQQSGAEVKKPGASVKVSCKASGYAFTSYLIHWIKQRPGQG |
| | | LEWIGVINPRSGDSHYNEKFKDRVTMTADQSISTAYMELSRLRSDD |
| | | TAVYYCARSDYGAYWGQGTLVTVSSASTKGPSVFPLAPSSKSTSG |
| | | GTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCP |
| | | PCPAPEFEGGPSVFLFPPKPKDTLMISRTPEVTCVVVDVSHEDPEV |
| | | KFNWYVDGVEVHNAKTKPREEQYNSTYRVVSVLTVLHQDWLNGK |
| | | EYKCKVSNKALPASIEKTISKAKGQPREPQVYTLPPSREEMTKNQV |
| | | SLTCLVKGFYPSDIAVEWESNGQPENNYKTTPPVLDSDGSFFLYS |
| | | KLTVDKSRWQQGNVFSCSVMHEALHNHYTQKSLSLSPG |
| 22 | aa | EIVLTQSPATLSVSPGERATLSCRASQSISNNLHWYQQKPGQAPR |
| | | LLIKYASQSAFGIPARFSGSGSGTEFTLTISSLQSEDFAVYFCQQSN |
| | | SWPHTFGQGTKLEIKRTVAAPSVFIFPPSDEQLKSGTASVVCLLNN |
| | | FYPREAKVQWKVDNALQSGNSQESVTEQDSKDSTYSLSSTLTLSK |
| | | ADYEKHKVYACEVTHQGLSSPVTKSFNRGEC |
| 23 | aa | YASQSIF |
| 24 | aa | QFAVVGPPGPILAMVGEDADLPCHLFPTMSAETMELRWVSSNLRQ |
| | | VVNVYADGKEVEDRQSAAYRGRTSILRDGITAGKAALRIHNVTASD |
| | | SGKYLCYFQDGDFYEKALVELKVAALGSDLHIDVKGYEDGGIHLEC |
| | | RSTGWYPQPQIRWSNDKGENIPAVEAPVFVDGVGLYAVAASVILR |
| | | GSSGEGVSCTIRSSLLGLEKTTSISIAGPFFRRAQSWIAALAGTLPV |
| | | LLLLLGGAGYFLWRQQEEKKTLFRKKKREQELRELAWSTVKQEKS |
| | | TREKLLEEIRWRSMQYTVLGERPSAYNEWKKALFKPADVILDPKTA |
| | | NPILLVSEDQRSVQRAKEPQDLPDNPERFDWHYCVLGCESFMSG |
| | | RHYWEVEVGDRKEWHIGVCSKNVQRKGWVKMTPENGSHIHTFLD |
| | | VSFSEPLYPVFRILTLEPTALTICPAPKEEESS |
| 25 | aa | LPCHLFPTMS AETMELKWVS S |

1-11. (canceled)

12. A method of treating a gastro-intestinal inflammatory disorder, in a human subject in need thereof, said method comprising administering a therapeutically efficient amount of an isolated anti-BTN3A antibody comprising means for binding specifically to BTN3A1 and inhibiting in vitro the degranulation of γδ T cells in co-culture with Daudi Burkitt's lymphoma cell lines with an IC50 of 10 nM or below.

13. The method of claim 12, wherein said isolated anti-BTN3A antibody is selected from the group consisting of:
(i) an antibody mAb1 having a heavy chain of SEQ ID NO:21 and a light chain of SEQ ID NO:22,
(ii) a variant of mAb1 having a heavy chain variable region (VH) of SEQ ID NO:7, and a light chain variable region (VL) of SEQ ID NO:8 but different constant regions,
(iii) a variant of mAb1 having HCDR1 of SEQ ID NO:1, HCDR2 of SEQ ID NO:2, HCDR3 of SEQ ID NO:3, LCDR1 of SEQ ID NO:4, LCDR2 of SEQ ID NO:5 and LCDR3 of SEQ ID NO:6 but different framework regions, or,
(iv) a variant of mAb1 which binds to the same epitope as mAb1, wherein said epitope comprises or essentially consists of SEQ ID NO:20, wherein said variant does not have HCDR1 of SEQ ID NO:1, HCDR2 of SEQ ID NO:2, HCDR3 of SEQ ID NO:3, LCDR1 of SEQ ID NO:4, LCDR2 of SEQ ID NO:23 and LCDR3 of SEQ ID NO:6.

14. The method of claim 12, wherein said isolated anti-BTN3A antibody is a variant of mAb1 having HCDR1 of SEQ ID NO:1, HCDR2 of SEQ ID NO:2, HCDR3 of SEQ ID NO:3, LCDR1 of SEQ ID NO:4, LCDR2 of SEQ ID NO:5 and LCDR3 of SEQ ID NO:6, wherein the VH amino acid sequence has at least 90% identity but less than 100% identity with SEQ ID NO:7, and the VL amino acid sequence has at least 90% identity but less than 100% identity with SEQ ID NO:8.

15. The method of claim 12, wherein said isolated anti-BTN3A antibody is a variant of mAb1 having HCDR1 of SEQ ID NO:1, HCDR2 of SEQ ID NO:2, HCDR3 of SEQ ID NO:3, LCDR1 of SEQ ID NO:4, LCDR2 of SEQ ID NO:5 and LCDR3 of SEQ ID NO:6, wherein the VH amino acid sequence has at least 95% identity but less than 100% identity with SEQ ID NO:7 and the VL amino acid sequence has at least 95% identity but less than 100% identity with SEQ ID NO:8.

16. The method of claim 12, wherein said isolated anti-BTN3A antibody comprises means for binding to the human BTN3A1 isoform with a K_{D} of 10 nM or less, as measured by surface plasmon resonance.

17. The method of claim 16, wherein said isolated anti-BTN3A antibody comprises means for binding to the human BTN3A1 isoform with a K_{D} between 1.10⁻¹¹ and 1.10⁻⁹ M.

18. The method of claim 12, wherein said isolated anti-BTN3A antibody is a functional variant of mAb1 which retains at least 90% of the affinity to BTN3A as measured by SPR assay, and comprises at least means for one or more of the following properties:
(i) inhibiting in vitro the degranulation of γδ T cells in co-culture with Daudi Burkitt's lymphoma cell lines with an EC50 of 10 nM or below, for example lnM or below, for example as determined in a CD107 degranulation assay by flow cytometry;
(ii) inhibiting substantially the phosphoantigen mediated activation, gut homing potential, proliferation and degranulation capacity of peripheral Vd2+ T cells from patients, for example as determined in an ex vivo assay with peripheral blood mononuclear cells (PBMCs) isolated from patients suffering from inflammatory bowel disease; or,
(iii) inhibiting substantially the phosphoantigen mediated activation or proliferation of gut Vd2+ T cells from gut biopsies of inflammatory bowel disease patients.

19. The method of claim 12, wherein said anti-BTN3A antibody is a human or humanized antibody.

20. The method of claim 12, wherein said anti-BTN3A antibody includes an IgG Fc region.

21. The method of claim 20, wherein said anti-BTN3A antibody includes a mutant IgG1 constant region having the amino acid substitutions L247F, L248E and P350S.

22. The method of claim 12, wherein said gastro-intestinal inflammatory disorder is inflammatory bowel disease.

23. A method of claim 12, wherein said gastro-intestinal inflammatory disorder is ulcerative colitis or Crohn's disease.

24. The method of claim 12, wherein a dose of 1 to 100 mg of isolated anti-BTN3A antibody is administered intravenously to the subject.

25. The method of claim 12, wherein said anti-BTN3A antibody is administered in combination, simultaneously or separately with an anti-inflammatory treatment.

26. The method of claim 12, wherein said anti-BTN3A antibody is administered in combination, simultaneously or separately with an anti-inflammatory treatment selected from anti-cytokine antibodies, anti-a4b7 integrin antibodies and JAK inhibitors.

27. The method of claim 12, wherein said anti-BTN3A antibody is administered in combination, simultaneously or separately with anti-TL-12, anti-TL-23 or anti-TNFa.
